(19) United States
(12) Patent Application Publication
(10) Pub. No.: US 2026/0201421 A1
(43) Pub. Date: Jul. 16, 2026

(54) SAFE HARBOUR LOCI FOR CELL ENGINEERING

(71) Applicants: Agency for Science, Technology and Research, Singapore (SG); National University of Singapore, Singapore (SG)

(72) Inventors: Arnaud Michel Yvon PERRIN, Singapore (SG); Efthymios MOTAKIS, Singapore (SG); Matias Ilmari AUTIO, Singapore (SG); Sik Yin Roger FOO, Singapore (SG)

(73) Assignees: Agency for Science, Technology and Research, Singapore (SG); National University of Singapore, Singapore (SG)

(21) Appl. No.: 19/136,822

(22) PCT Filed: Dec. 7, 2022
(86) PCT No.: PCT/SG2022/050888

§ 371 Date: Jun. 6, 2025

(51) Int. Cl. C12N 15/90; C12N 5/0735; C12N 5/074; C12N 5/0783; C12N 9/22; C12N 15/10; C12N 15/113; C12N 15/85

(52) U.S. Cl. CPC C12N 15/907; C12N 5/0606; C12N 5/0636; C12N 5/0646; C12N 5/0696; C12N 9/226; C12N 15/102; C12N 15/113; C12N 15/85; C12N 2310/20; C12N 2510/00; C12N 2830/34

(57) ABSTRACT

The present invention relates generally to the field of molecular biology. In particular, the specification teaches an engineered cell comprising at least one heterologous polynucleotide inserted into a safe harbour locus.

### TECHNICAL FIELD

The present invention relates generally to the field of molecular biology. In particular, the specification teaches an engineered cell comprising at least one heterologous polynucleotide inserted into a safe harbour locus.

### BACKGROUND

Controlled expression of transgenes in cells is essential for both therapeutic and research purposes. Traditionally such transgene expression has been accomplished via viral targeting and integration in the host genome in a semi-random fashion. The randomly integrated transgenes may undergo silencing or disrupt endogenous genes, potentially giving rise to malignancies. The advent of genomic targeting technologies, such as zinc finger nucleases and CRISPR/Cas9, has enabled directed integration of transgenes into a cell of interest. The ideal integration sites for the foreign genetic material are “safe harbour” sites, which allow for controlled expression of a transgene without perturbing endogenous gene expression patterns. To date, a number of sites in the human genome have been used for directed integration, including the AAVS1, CCR5 and human-orthologous Rosa26 sites. However, these sites reside in highly gene-rich regions and, in the case of AAVS1, the site resides within a gene transcription unit. Furthermore, all of these loci have known oncogenes in their proximity (<300 kb). Thus, their utilization in a clinical setting would require extensive further safety data. Furthermore variable transgene expression and silencing has been reported for AAVS1 in hepatocytes and cardiomyocytes.

It would be desirable to overcome or ameliorate at least one of the above-described problems, or at least to provide a useful alternative.

### SUMMARY

Disclosed herein is an engineered cell comprising at least one heterologous polynucleotide inserted into a safe harbour locus listed in Table 1.

Disclosed herein is a composition comprising the engineered cell as defined herein and a pharmaceutical excipient.

Disclosed herein is a method of editing a cell, the method comprises inserting at least one heterologous polynucleotide into a safe harbour locus in the cell, wherein the safe harbour locus is a safe harbour locus listed in Table 1.

Disclosed herein is a method of editing a cell, the method comprising contacting the cell with one or more gRNAs, one or more Cas9 endonucleases and at least one heterologous polynucleotide, wherein the one or more gRNAs and Cas9 endonucleases facilitate the insertion of the at least one heterologous polynucleotide into chromosomal DNA within a safe harbor locus, wherein the safe harbor locus is a safe harbour locus listed in Table 1.

Disclosed herein is a method of preparing a master clonal cell line, the method comprises inserting at least one heterologous polynucleotide into a safe harbour locus in a cell, wherein the safe harbour locus is a safe harbour locus listed in Table 1.

Disclosed herein is a guide ribonucleic acids (gRNA) for editing a cell at a safe harbor locus, wherein the gRNA comprises any one of the sgRNA sequences in Table 2.

Disclosed herein is a method of treating a subject having or at risk of having a disease, the method comprising administering to the subject an effective amount of the engineered cell as defined herein, or the composition as defined herein, to the subject.

### BRIEF DESCRIPTION OF THE DRAWINGS

Embodiments of the present invention will now be described, by way of non-limiting example, with reference to the drawings in which:

FIG. 1 shows: A) Schematic representation of the computational workflow for defining candidate genomic safe harbour (GSH) loci. B) CIRCOS plot summarising computational search results. Ring 1: chromosome ideograms; ring 2: orange bars indicating safe sites; ring 3: blue bars indicating active regions; ring 4: candidate sites within active regions, red bars site failed BLAT screening, black bars site passed BLAT screening. C) Locations of candidate GSH targeted in vitro. Blue labels: targeted clone established; green labels: no clone established.

FIG. 2 shows: A) Schematic representation of CRISPR/Cas9 plasmid (pMIA3) and homology directed repair donor (pMIA4.721) used for targeting with functional components annotated. B) Schematic of integrated landing pad expression construct. Positions of primers for junction-PCR as well as of ddPCR assay are indicated. Representative junction-PCR Sanger sequencing reads from Pansio-1 targeted clones shown in expanded view. C) Log 2-FC of mRNA expression levels against un-targeted H1 hESC samples for the nearest genes of Pansio-1, Olônne-18, and Keppel-19 candidate GSH. Evaluated samples: H1=un-targeted hESC, CTRL=H1 cells non-GSH targeted, Pansio-1=landing pad construct integrated to Pansio-1 GSH in H1 hESC, Olônne-18=landing pad construct integrated to Olônne-18 GSH in H1 hESC and Keppel-19=landing pad construct integrated to Keppel-19 GSH in H1 hESC. Box plots representing 95% confidence intervals of mean log 2-FC. Nearest gene for each GSH indicated in orange. Individual data points shown in pink with P-value for each comparison shown above. D) Volcano plots of RNA-seq analysis against un-targeted H1 hESC. Samples analysed as in C). Differentially expressed (DE) genes with FDR≤0.01 and |log FC|≥1 in pink, genes with |log FC|≥1 in green, genes with FDR≤0.01 in blue, others in grey. E) Venn-diagrams illustrating the overlap of DE genes between un-targeted H1 hESC, non-GSH targeted H1 hESC and the three GSH targeted H1 hESC lines. F) Representative images of metaphase spreads used for karyotyping the GSH targeted cell lines.

FIG. 3 shows: A) Schematic representation of integrase expression construct (pMIA22) and transposon donor construct (pMIA10.5). B) Schematic of landing pad construct with integrated Clover transgene. C) Representative immunofluorescence images of Clover-integrated GSH H1 cells. DAPI=nuclear staining with 4′,6-diamidino-2-phenylindole, Clover=fluorescence from Clover transgene, OCT3/4=antibody staining against OCT3/4, Overlay=overlay of the three imaged channels. D) As in C) apart from antibody staining against SOX2. E) Histograms of flow cytometry analysis for FITC-A channel of un-targeted H1 hESC, and the three GSH targeted hESC lines over 15 passages. Percentages of FITC-A positive cells according to the indicated gating. F) Representative immunofluorescence images of Clover-integrated GSH HI cells differentiated to neuronal-like cells. Channels imaged as in C) apart from antibody staining against Tuj1. G) As in F) for cells differentiated to hepatocyte-like cells, antibody staining against AFP. H) As in F) for cells differentiated to cardiomyocyte-like cells, antibody staining against sarcomeric α-ACTININ. Scale bars for all immunofluorescence images equal to 150 μm.

FIG. 4 shows screenshots of Hi-C interaction matrices from H1 hESC for each of the shortlisted GSH candidate loci. TADs are indicated by the “pyramids” of high interaction observed in the Hi-C matrices. UCSC genome browser track annotating the candidate GSH (targeted GSH in pink), GENCODE v36 and H3K27Ac mark from ENCODE shown below.

FIG. 5 shows PCR gel images of junction PCR and wild type allele PCR reactions for screened clones.

FIG. 6 shows representative images of Pansio-1 line immunofluorescence staining with AF594 secondary antibody alone. DAPI=nuclear staining with 4′,6-diamidino-2-phenylindole, Clover=fluorescence from Clover transgene, AF594=staining with secondary antibody alone, Overlay=overlay of the three imaged channels.

FIG. 7 shows a schematic of the pMIA22 plasmid and schematic of recombinase-mediated cassette exchange (RMCE) components and efficiency test experiment.

FIG. 8 shows: A) Relative fluorescence data from triplicate experiment of transfection of HEK cells with GFP donor, expression acceptor and different integrase constructs. Fluorescence intensity is normalised to untransfected cells. B) Example of flow cytometry data of HEK cells transfected with GFP donor, expression construct and different integrase constructs

### DETAILED DESCRIPTION

The present specification teaches an engineered cell comprising at least one heterologous polynucleotide inserted into a safe harbour locus listed in Table 1.

Without being bound by theory, the inventors have relied on a number of criteria for identifying a safe habour locus. These include the following: a) the locus is not in an ultra-conserved region or in a transcription unit; b) it is in a region not less than 2 kb away at both ends from a DNase hypersensitivity cluster; c) it is in a region not less than 50 kb away at both ends from a transcription start site; d) it is in a region not less than 100 kb away at both ends from a sequence for a long non-coding RNA; e) it is in a region not less than 300 kb away at both ends from a cancer-related gene or a microRNA-coding sequence; f) it is associated with at least one ubiquitously-expressed, low-variance housekeeping gene; and g) it is in an active chromosomal compartment.

In some embodiments, the safe habour locus is Chrl 1:113339961-113340514, Chrl 18:56534775-56536439 or Chrl 19:5400761-5402139. In some embodiments, the safe habour locus is Chrl 1:113339961-113340514. In some embodiments, the safe harbour locus is Chrl 18:56534775-56536439. In some embodiments, the safe harbour locus is Chrl 19:5400761-5402139.

In some embodiments, the safe habour locus is selected from Chrl 1:113339961-113340514, Chrl 18:56534775-56536439 or Chrl 19:5400761-5402139. In some embodiments, the safe habour locus is selected from Chrl 1:113339961-113340514. In some embodiments, the safe harbour locus is selected from Chrl 18:56534775-56536439. In some embodiments, the safe harbour locus is selected from Chrl 19:5400761-5402139.

In some embodiments, the integration site is in Chrl 1:113339961-1133400061, Chrl 1:1133400061-1133400161, Chrl 1:1133400161-1133400261, Chrl 1:1133400261-1133400361, Chrl 1:1133400361-1133400461 or Chrl 1:1133400461-1133400514. In some embodiments, the safe habour locus is Chrl 1:113340393-113340412. In some embodiments, the safe habour locus is Chrl 1:113340395-113340396.

In some embodiments, the safe habour locus is Chrl 18:56534775-56534875, Chrl 18:56534875-56534975, Chrl 18:56534975-56535075, Chrl 18:56535075-56535175, Chrl 18:56535175-56535275, Chrl 18:56535275-56535375, Chrl 18:56535375-56535475, Chrl 18:56535475-56535575, Chrl 18:56535575-56535675, Chrl 18:56535675-56535775, Chrl 18:56535775-56535875, Chrl 18:56535875-56535975, Chrl 18:56535975-56536075, Chrl 18:56536075-56536175, Chrl 18:56536175-56536275, Chrl 18:56536275-56536375 or Chrl 18:56536375-56536439. In some embodiments, the safe habour locus is Chrl 18:56535738-56535757. In some embodiments, the safe habour locus is Chrl 18:56535740-56535741.

In some embodiments, the safe habour locus is Chrl 19:5400761-5400861, Chrl 19:5400861-5400961, Chrl 19:5400961-5401061, Chrl 19:5401061-5401161, Chrl 19:5401161-5401261, Chrl 19:5401261-5401361, Chrl 19:5401361-5401461, Chrl 19:5401461-5401561, Chrl 19:5401561-5401661, Chrl 19:5401661-5401761, Chrl 19:5401761-5401861, Chrl 19:5401861-5401961, Chrl 19:5401961-5402061 or Chrl 19:5402061-5402139. In some embodiments, the safe habour locus is Chrl 19:5400904-5400923. In some embodiments, the safe habour locus is Chrl 19:5400906-5400907.

In some embodiments, the safe habour locus is Chrl 1:113340393-113340412, Chrl 18:56535738-56535757 or Chrl 19:5400904-5400923.

In some embodiments, the safe habour locus is Chrl 1:113340395-113340396, Chrl 18:56535740-56535741 or Chrl 19:5400906-5400907.

In some embodiments, the introduction of a transgene into a safe habour locus as defined herein does not alter the expression of a pluripotency marker (such as OCT3/4 and/or SOX2) in a cell. In some embodiments, the introduction of a transgene into a safe harbour locus as defined herein does not induce differentiation of the cell. In some embodiments, the expression of the transgene is maintained even after the cell has been induced to undergo differentiation into other cell types.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.

As used herein, the term “locus” refers to a specific, fixed physical location on a chromosome.

As used herein, the term “target locus” refers to a locus on a chromosome within which a safe harbour locus can be used for the insertion of a heterologous polynucleotide. A target locus can consist of multiple potential integration sites for polynucleotide insertion. Examples of target loci are provided in Table 1. The notation used in Table 1 refers to the genomic region of the target locus, defined by the chromosome of the target locus and the coordinate range for that target locus. For example, Chrl 1:113339961-113340514 refers to a target locus on Chrl 1 (chromosome 1) starting from coordinate 113339961 and ending with coordinate 113340514.

The term “genomic safe harbour” or “GSH” or “safe harbour locus” refers to a locus at which genes or genetic elements can be incorporated without disruption to expression or regulation of adjacent genes. These safe harbour loci are also referred to as safe harbour sites (SHS). As used herein, a safe harbour locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site.

As used herein, the term “insert” refers to a nucleotide sequence that is integrated (inserted) at a safe harbour site. The insert can be used to refer to the genes or genetic elements that are incorporated at the safe harbour site using, for example, homology-directed repair (HDR) CRISPR/Cas9 genome-editing or other methods for inserting nucleotide sequences into a genomic region known to those of ordinary skill in the art.

The “CRISPR/Cas” system refers to a widespread class of bacterial systems for defense against foreign nucleic acids. CRISPR/Cas systems are found in a wide range of eubacterial and archaeal organisms. CRISPR/Cas systems include type I, II, and III subtypes. Wild-type type II CRISPR/Cas systems utilize an RNA-mediated nuclease, Cas9, in complex with targeting and activating RNA to recognize and cleave foreign nucleic acid. Guide RNAs (gRNAs) having the activity of both a targeting RNA and an activating RNA are also known in the art. In some cases, such dual activity guide RNAs are referred to as a single guide RNAs (sgRNAs).

Cas9 homologs are found in a wide variety of eubacteria, including, but not limited to bacteria of the following taxonomic groups: Actinobacteria, Aquificae, Bacteroidetes, Chlorobi, Chlamydiae, Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 une; 9(6): 467-477; Hou, et al., Proc Natl Acad Sci USA. 2013 Sep. 24; 110(39): 15644-9; Sampson et al., Nature. 2013 May 9; 497(7448): 254-7; and Linek, et al., Science. 2012 Aug. 17; 337(6096): 816-21. The Cas9 nuclease domain can be optimized for efficient activity or enhanced stability in the host cell.

As used herein, the term “Cas9” refers to an RNA-mediated nuclease (e.g., of bacterial or archeal origin, or derived therefrom). Exemplary RNA-mediated nucleases include the foregoing Cas9 proteins and homologs thereof, and include but are not limited to, CPF1 (See, e.g., Zetsche et al., Cell, Volume 163, Issue 3, p 759-771, 22 Oct. 2015). Similarly, as used herein, the term “Cas9 ribonucleoprotein” complex and the like refers to a complex between the Cas9 protein, and a crRNA, the Cas9 protein and a trans-activating crRNA (tracrRNA), the Cas9 protein and a single guide RNA, or a combination thereof (e.g., a complex containing the Cas9 protein, a tracrRNA, and a crRNA guide RNA).

Provided herein is a guide ribonucleic acid (gRNA) for editing a cell at a safe harbor locus, wherein the gRNA targets any one of the sequences in Table 2. Also provided herein is a construct comprising a nucleic acid sequence encoding a gRNA as defined herein. In some embodiments, there is provided a vector comprising a construct, wherein the construct comprises a nucleic acid sequence encoding a gRNA as defined herein.

As used herein, the term “ex vivo” generally includes experiments or measurements made in or on living tissue, preferably in an artificial environment outside the organism, preferably with minimal differences from natural conditions.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can encode miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

The term “chromosomal landing pad” (or simply “landing pad”) refers to a site-specific recognition sequence or a site-specific recombination site (e.g., an attP site) that is stably integrated into the genome of a host cell. In particular, the site-specific recognition sequence or recombination site is inserted into the host genome at one or more safe harbour loci as disclosed herein. Presence in the host genome of the heterologous site-specific recombination sequence allows a recombinase to mediate site-specific insertion of a heterologous polynucleotide or a transgene into the host genome. Typically, in order to integrate into the landing pad, the heterologous polynucleotide or transgene is attached to a cognate recognition sequence or recombination site (e.g., an attB site if the inserted site-specific recombination site is an attP site) that is also recognized by the recombinase.

In some embodiments, the heterologous polynucleotide comprises one or more site-specific recombination sequences. The site-specific recombination sequence may be a recognition sequence recognized by a site-specific recombinase. For example, the recognition sequence may comprise an attP site or attB site that is recognized by a BxbI integrase.

As used herein, “site-specific recombinase” refers to a family of enzymes that mediate the site-specific recombination between specific DNA sequences recognized by the enzymes. Examples of site-specific recombinase include, without limitation, Cre recombinase, Flp recombinase, the lambda integrase, gamma-delta resolvase, Tn3 resolvase, Sin resolvase, Gin invertase, Hin invertase, Tn5044 resolvase, Tn3 transposase, sleeping beauty transposase, IS607 transposase, BxbI integrase, wBeta integrase, BL3 integrase, phiR4 integrase, A118 integrase, TGI integrase, MRU integrase, phi370 integrase, SPBc integrase, TP901-1 integrase, phiRV integrase, FC1 integrase, K38 integrase, phiBTl integrase and phiC31 integrase. In certain embodiments, the site-specific recombinase is a uni-directional recombinase. As used herein, “uni-directional recombinases” refer to recombinase enzymes whose recognition sites are destroyed after recombination has taken place. In other words, the sequence recognized by the recombinase is changed into one that is not recognized by the recombinase upon recombination mediated by the recombinase, and the continued presence of the recombinase cannot reverse the previous recombination event. Examples of uni-directional recombinase include, without limitation, phiC31 integrase and Bxbl integrase.

Provided herein is also a nucleic acid sequence that is codon-optimized for Bxbl integrase expression. Provided herein is a nucleic acid sequence having at least 70% (including at least 80%, 90%, 95% or 99%) sequence identity to SEQ ID NO: 136. In some embodiments, there is provided a construct comprising a nucleic acid sequence having at least 70% (including at least 80%, 90%, 95% or 99%) sequence identity to SEQ ID NO: 136. In some embodiments, there is provided a construct comprising a nucleic acid sequence having at least 70% (including at least 80%, 90%, 95% or 99%) sequence identity to SEQ ID NO: 137. Provided herein is a vector comprising a construct as defined herein.

Provided herein is a method of inserting a polynucleotide sequence into a genome of an engineered cell, comprising introducing into the cell: a) a nucleic acid sequence encoding a BxbI integrase as defined herein; and b) a polynucleotide comprising a heterologous polynucleotide flanked by one or more (e.g. two) site specific recombination sequence (e.g. attP site or attB site) recognised by the BxbI integrase, wherein the engineered cell comprises a polynucleotide comprising one or more (e.g. two) site specific recombination sequence integrated into a locus in the genome of the engineered cell.

As used herein, the term “transgene” refers to a polynucleotide that has been transferred naturally, or by any of a number of genetic engineering techniques into a cell. It is optionally translated into a polypeptide. It is optionally translated into a recombinant protein. A “recombinant protein” is a protein encoded by a gene that has been cloned in a system that supports expression of the gene and translation of messenger RNA. The recombinant protein can be a therapeutic agent, e.g. a protein that treats a disease or disorder disclosed herein. As used, transgene can refer to a polynucleotide that encodes a polypeptide. A transgene can also refer to a non-encoding sequence, such as but not limited, to shRNAs, miRNAs, and miRs.

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and anti sense DNA, and spliced or unspliced mRNA, rRNA, tRNA and inhibitory DNA or RNA (RNAi, e.g., small or short hairpin (sh) RNA, microRNA, small or short interfering (si)RNA, trans-splicing RNA, or antisense RNA). Polynucleotides include naturally occurring, synthetic, and intentionally altered or modified polynucleotides as well as analogues and derivatives. Polynucleotides can be single, double, or triplex, linear or circular, and can be of any length.

The terms “protein,” “polypeptide,” and “peptide” are used herein interchangeably.

As used herein, the term “developmental cell states” refers to, for example, states when the cell is inactive, actively expressing, differentiating, senescent, etc. developmental cell state may also refer to a cell in a precursor state (e.g., a T cell precursor or T cell progenitor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is an RNA molecule. When present as an RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “operably linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue-specific or developmentally-regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example U.S. application Ser. No. 15/715,068, the disclosures of which are herein incorporated by reference in their entirety.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

As used herein, the term “effective amount” refers to the amount of a compound e.g., a compositions described herein, cells described herein) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

In some embodiments, the present disclosure contemplates inserts that comprise one or more transgenes. The transgene can encode a therapeutic protein, an antibody, a peptide, a suicide gene, an apoptosis gene or any other gene of interest. The safe harbour loci identified herein allow for transgene integration that results in, for example, enhanced therapeutic properties. These enhanced therapeutic properties, as used herein, refer to an enhanced therapeutic property of a cell when compared to a typical cell of the same normal cell type. For example, an NK cell having “enhanced therapeutic properties” has an enhanced, improved, and/or increased treatment outcome when compared to a typical, unmodified and/or naturally occurring NK cell. The therapeutic properties of cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the safe harbour site. In some embodiments, the insert size comprises at least about 100, 200, 300, 400 or 500 base pairs. In some embodiments, the insert size comprises about 500 nucleotides or base pairs. In some embodiments, the insert size comprises up to 0.5, 0.6, 0.7, 0.8, 0.9, 1, 2, 3, 4, 5, 6, 7, 8, 9, 10, 11, 12, 13, 14, 15, 16, 17, 18, 19, 20 kbp (kilo base pairs) or the sizes in between. In some embodiments, the insert size is greater than 0.5, 0.6, 0.7, 0.8, 0.9, 1, 2, 3, 4, 5, 6, 7, 8, 9, 10, 11, 12, 13, 14, 15, 16, 17, 18, 19, 20 kbp or the sizes in between. In some embodiments, the insert size is within the range of 3-15 kbp or is any number in that range. In some embodiments, the insert size is within the range of 1.5-10 kbp or is any number in that range. In some embodiments, the insert size is within the range of 1.5-15 kbp or is any number in that range. In some embodiments, the insert size is within the range of 0.5-20 kbp or is any number in that range. In some embodiments, the insert size is 0.5-10, 0.6-10, 0.7-10, 0.8-10, 0.9-10, 1-10, 2-10, 3-10, 4-10, 5-10, 6-10, 7-10, 8-10, 9-10 kbp. In some embodiments, the insert size is 0.5-11, 0.6-11, 0.7-11, 0.8-11, 0.9-11, 1-11, 2-11, 3-11, 4-11, 5-11, 6-11, 7-11, 8-11, 9-11, or 10-11 kbp. In some embodiments, the insert size is 0.5-12, 0.6-12, 0.7-12, 0.8-12, 0.9-12, 1-12, 2-12, 3-12, 4-12, 5-12, 6-12, 7-12, 8-12, 9-12, 10-12, or 11-12 kbp. In some embodiments, the insert size is 0.5-13, 0.6-13, 0.7-13, 0.8-13, 0.9-13, 1-13, 2-13, 3-13, 4-13, 5-13, 6-13, 7-13, 8-13, 9-13, 10-13, 11-13, or 12-13 kbp. In some embodiments, the insert size is 0.5-14, 0.6-14, 0.7-14, 0.8-14, 0.9-14, 1-14, 2-14, 3-14, 4-14, 5-14, 6-14, 7-14, 8-14, 9-14, 10-14, 11-14, 12-14 or 13-14 kbp. In some embodiments, the insert size is 0.5-15, 0.6-15, 0.7-15, 0.8-15, 0.9-15, 1-15, 2-15, 3-15, 4-15, 5-15, 6-15, 7-15, 8-15, 9-15, 10-15, 11-15, 12-15, 13-15, or 14-15 kbp. In some embodiments, the insert size is 0.5-16, 0.6-16, 0.7-16, 0.8-16, 0.9-16, 1-16, 2-16, 3-16, 4-16, 5-16, 6-16, 7-16, 8-16, 9-16, 10-16, 11-16, 12-16, 13-16, 14-16 or 15-16 kbp. In some embodiments, the insert size is 0.5-17, 0.6-17, 0.7-17, 0.8-17, 0.9-17, 1-17, 2-17, 3-17, 4-17, 5-17, 6-17, 7-17, 8-17, 9-17, 10-17, 11-17, 12-17, 13-17, or 14-17, 15-17 or 16-17 kbp. In some embodiments, the insert size is 0.5-18, 0.6-18, 0.7-18, 0.8-18, 0.9-18, 1-18, 2-18, 3-18, 4-18, 5-18, 6-18, 7-18, 8-18, 9-18, 10-18, 11-18, 12-18, 13-18, 14-18, 15-18, 16-18 or 17-18 kbp. In some embodiments, the insert size is 0.5-19, 0.6-19, 0.7-19, 0.8-19, 0.9-19, 1-19, 2-19, 3-19, 4-19, 5-19, 6-19, 7-19, 8-19, 9-19, 10-19, 11-19, 12-19, 13-19, 14-19, 15-19, 16-19, 17-19, or 18-19 kbp. In some embodiments, the insert size is 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a safe harbour site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprise a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), syn-notch, CARs, mAbs, etc. In some embodiments, the inserts are RNAi molecules, including, but not limited to, miRNAs, siRNA, shRNAs, etc. In some embodiments, the insert is a human sequence. In some embodiments, the insert is chimeric. In some embodiments, the insert is a multi-gene/multi-module therapeutic cassette. A multi-gene/multi-module therapeutic cassette refers to an insert or cassette having one or more than one gene, other exogenous protein coding sequences, non-coding RNAs, transcriptional regulatory elements, and/or insulator sequences, etc.

Various cell types are contemplated as having the safe harbour sites in the present disclosure. A cell comprising a safe harbour site and/or a cell comprising an insert at a safe harbour site as described in the present disclosure can be referred to as an engineered cell. The cell can be a mammalian cell, for example, a human cell. In some embodiments, the engineered cell is primary cell, a stem cell, an immune cell or a cell line. A primary cell may be a fibroblast or any primary somatic cell, non-limiting examples of which include epithelial, endothelial, neuronal, adipose, cardiac, skeletal muscle, immune, hepatic, splenic, lung, circulating blood cells, gastrointestinal, renal, bone marrow, and pancreatic cells. Non-limiting examples of immune cells that are contemplated in the present disclosure include T cell, B cell, natural killer (NK) cell, NKT/iNKT cell, macrophage, myeloid cell, and dendritic cells. In one embodiment, the immune cell is a T cell or an NK cell. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cell is a T cell, a NK cell, an ESC, an iPSC or a fibroblast. In some embodiments, the engineered cells are cell lines grown in vitro (e.g. deliberately immortalized cell lines, cancer cell lines, etc.). For example, a cell line may be a HEK, PER-C6, HKB-11 or HuH-7 cell line.

In some embodiments an engineered cell contains a chromosomal landing pad at one or more safe harbour sites. The chromosomal landing pad allows the site-specific insertion of a heterologous polynucleotide or a transgene into the cell.

In some embodiments an engineered cell is further differentiated or reprogrammed without altering any of the safe harbour loci of this disclosure that it contains, or any heterologous polynucleotide or chromosomal landing pad that is present at a safe harbour locus in the cell. “Reprogramming” refers to the process of altering a cell phenotype from a somatic cell or progenitor cell phenotype to a stem cell-like phenotype. A somatic or progenitor cell may be reprogrammed by the introduction of nucleic acid sequences encoding stem cell-associated genes into the cell. In general, these nucleic acids are introduced using viral vectors (such as Sendai viral vectors) and expression of the gene products results in cells that are morphologically and biochemically similar to pluripotent stem cells (e.g., embryonic stem cells). Cell reprogramming is known in the art and is described in, e.g. Aydin et al., Ann Rev Cell Develop Biol, Vol. 35:433-452, 2019. An engineered stem cell of this disclosure or an engineered cell that has been reprogrammed to a stem cell may be induced to differentiate to obtain a desired cell type according to known methods to differentiate stem cells. For example, stem cells may be induced to differentiate into hematopoietic stem cells, muscle cells, cardiac muscle cells, liver cells, pancreatic cells, cartilage cells, epithelial cells, urinary tract cells, nervous system cells (e.g., neurons) etc., by culturing such cells in differentiation medium and under conditions which provide for cell differentiation. Medium and methods which result in the differentiation of embryonic stem cells are known in the art as are suitable culturing conditions.

The methods for integrating the inserts at the safe harbour sites can be viral or non-viral delivery techniques.

In some embodiments, the nucleic acid sequence is inserted into the genome of the engineered cell by introducing a vector, for example, a viral vector, comprising the nucleic acid. Examples of viral vectors include, but are not limited to, adeno-associated viral (AAV) vectors, retroviral vectors or lentiviral vectors. In some embodiments, the lentiviral vector is an integrase-deficient lentiviral vector.

In some embodiments, the nucleic acid sequence is inserted into the genome of the T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbour loci include, without limitation, homology-dependent engineering using nucleases and homology independent targeted insertion using Cas9. In some embodiments, the non-viral delivery method comprises electroporation.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

In some embodiments a transgene inserted at a safe harbour locus disclosed herein remains intact over cell passages in vitro or over cell division cycles in vitro or in vivo. The transgene may be operably linked to an endogenous or exogenous or synthetic promoter.

### Immune Cell Therapy

Chimeric antigen receptor (CAR) immune cells are immune cells that have been genetically engineered to produce an artificial cell surface receptor for use in immunotherapy. Chimeric antigen receptors are receptor proteins that have been engineered to confer immune cells with the ability to target a specific protein. The genetic modification of lymphocytes (e.g. T cells, NK cells, NKT cells, iNKT cells) or macrophages by incorporation of, for example, CARs, and administration of the engineered cells to a subject is an example of “adoptive cell therapy”. As used herein, the term “adoptive cell therapy” refers to cell-based immunotherapy for transfusion of autologous or allogeneic lymphocytes or macrophages. In this CAR therapy approach, cells are expanded and cultured ex vivo and genetically modified, prior to transfusion.

The expression of CARs allows the engineered immune cells to target and bind specific proteins, for example, tumor antigens. In CAR therapy, immune cells are harvested from a subject—they can be autologous immune cells from the subject own blood or from a donor that will not be receiving the CAR therapy. Once isolated, the immune cells are genetically modified with a CAR, expanded ex vivo, and administered to the subject (i.e. patient) by, e.g. infusion.

The CARs may be introduced into the immune cells using, for example, a viral technique (e.g., retroviral integration) or site-specific technique. With site specific integration of the transgenes (e.g. CARs), the transgenes may be targeted to a safe harbor locus. Examples of site-specific techniques for integration into the safe harbor loci include, without limitation, homology-dependent engineering using nucleases and homology independent targeted insertion using Cas9.

The engineered CAR immune cells have applications to immune-oncology. The CAR, for example, can be selected to target a specific tumor antigen. Examples of cancers that can be effectively targeted using CAR T cells are blood cancers. In some embodiments, CAR T cell therapy can be used to treat solid tumors.

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behaviour (e.g. for therapeutic purposes).

Currently available genome editing tools include zinc finger nucleases (ZFN) and transcription activator-like effector nucleases (TALENs) to incorporate genes at safe harbor loci. The DICE (dual integrase cassette exchange) system utilizing phiC31 integrase and BxbI integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at safe harbor loci.

### Crispr-Cas Gene Editing

One effective example of gene editing is the Crisp-Cas approach (e.g. Crispr-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540(7631), 144-149, the relevant disclosures of which are herein incorporated in their entirety).

Methods for conducing gene editing using CRISPR-Cas approaches are known to those of ordinary skill in the art. (See, for example, U.S. application Ser. Nos. 16/312,676, 15/303,722, and 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example U.S. application Ser. No. 15/715,068, the disclosures of which are herein incorporated by reference in their entirety.

### Therapeutic Applications

Provided herein is a method of treating a subject having or at risk of having a disease, the method comprising administering to the subject an effective amount of the engineered cell as defined herein, or the composition as defined herein, to the subject.

For therapeutic applications, the engineered cells, populations thereof, or compositions thereof are administered to a subject, generally a mammal, generally a human, in an effective amount.

The engineered cells may be administered to a subject by infusion e.g., continuous infusion over a period of time) or other modes of administration known to those of ordinary skill in the art.

The engineered cells provided herein can be administered as part of a pharmaceutical compositions. In some embodiments, the present disclosure provides compositions comprising a guide RNA of the present disclosure. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. In some embodiments, the engineered cells are immune cells. In some embodiments, the engineered cells are lymphocytes. In some embodiments, the engineered cells are T cells or T cell progenitors. In some embodiments, the engineered cells are NK cells. The engineered cells, compositions and methods of the present disclosure are useful for therapeutic applications such as immune cell therapy, TCR T cell therapy and regenerative medicine. In some embodiments, the insertion of a sequence encoding a transgene within a safe harbor locus maintains the TCR expression relative to instances when there is no insertion and enables transgene expression while maintaining TCR function.

Various diseases treated using the engineered cells, populations thereof, or compositions thereof are provided herein. Non-limiting examples of such diseases include alopecia areata, autoimmune hemolytic anemia, autoimmune hepatitis, cancer, dermatomyositis, diabetes (type 1), certain juvenile idiopathic arthritis, glomerulonephritis, Graves' disease, Guillain Valley Syndrome, idiopathic thrombocytopenia purpura, myasthenia gravis, certain myocarditis, multiple sclerosis, pemphigus/pemphigoid, pernicious anemia, polyarteritis nodosa, polymyositis, primary bile With cirrhosis, psoriasis, rheumatoid arthritis, scleroderma/systemic sclerosis, Sjogren's syndrome, systemic lupus erythematosus, certain thyroiditis, certain uveitis, vitiligo, multiple vasculitis (Wegener)); autoimmune disorders including, but not limited to, granulomatosis; hematopoietic tumors including but not limited to acute and chronic leukemia, lymphoma, multiple myeloma and myelodysplastic syndrome; tumors of the prostate, breast, lung, colon, uterus, skin, liver, bone, pancreas, ovary, testis, bladder, kidney, head, neck, stomach, cervix, rectum, larynx, or esophagus solid tumors; HIV (human immunodeficiency virus) related disorders, RSV (respiratory syncytial virus) related disorders; EBV (Epstein-Barr virus) related disorders; CMV (cytomegalovirus) related disorders; and infectious diseases including, but not limited to, adenovirus-related disorders and BK polyomavirus-related disorders.

Cancers that can be treated with the engineered cells (e.g., CAR T-cells) of the present disclosure, populations thereof, or compositions thereof include blood cancers. In some embodiments, the cancer treated using the engineered cells described herein, populations thereof, or compositions thereof is a hematologic malignancy or leukemia. In some embodiments, the engineered cells (e.g., CAR T-cells) described herein, populations thereof, or compositions thereof are used for the treatment of acute lymphoblastic leukemia (ALL) or diffuse large B-cell lymphoma (DLBCL). In some embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia. Examples of cancers treatable using the engineered cells (e.g., CAR T-cells) described herein include, without limitation, breast cancer, ovarian cancer, esophageal cancer, bladder or gastric cancer, salivary duct carcinoma, salivary duct carcinomas, adenocarcinoma of the lung or aggressive forms of uterine cancer, such as uterine serous endometrial carcinoma. In some other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinomajejunum cancer joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatinsecreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some embodiments, the present disclosure provides methods of treating a subject in need of treatment by administering to the subject a composition comprising any of the engineered cells described herein.

As used, the terms “treat,” “treatment,” and the like refer generally to obtaining a desired pharmacological and/or physiological effect. That effect is preventive in terms of complete or partial prevention of the disease and/or therapeutic in terms of partial or complete cure of the disease and/or adverse effects resulting from the disease. The term “treatment”, as used herein, encompasses any treatment of a disease in a subject (e.g., mammal, e.g., human). Treatment may also refer to the administration of the engineered cells provided herein to a subject that is susceptible to the disease but has not yet been diagnosed as suffering from it, including preventing the disease from occurring; inhibiting disease progression; or reducing the disease (i.e., causing a regression of the disease). Further, treatment may stabilize or reduce undesirable clinical symptoms in subjects (e.g., patients). The cells provided herein populations thereof, or compositions thereof may be administered before, during or after the occurrence of the disease or injury.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition. In certain embodiments, a subject in need of cell therapy includes, but is not limited to, a bone marrow transplant or stem cell transplant candidate, a subject who has received chemotherapy or radiation therapy, a hyperproliferative disease or cancer (e.g., a hematopoietic system), a subject having or at risk of developing a hyperproliferative disease or cancer), a subject having or at risk of developing a tumor (e.g., solid tumor), viral infection or virus. It is also intended to encompass subjects suffering from or at risk of suffering from a disease associated with an infection.

In some embodiments, the present disclosure provides a composition of the present disclosure along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

### Combination Therapies

In some embodiments, an engineered cells of the present disclosure or composition thereof is administered with at least one additional therapeutic agent. Any suitable additional therapeutic agent may be administered with an engineered cell provided herein, populations thereof, or compositions thereof. In some aspects, the additional therapeutic agent is selected from radiation, an ophthalmologic agent, a cytotoxic agent, a chemotherapeutic agent, a cytostatic agent, an anti-hormonal agent, an immunostimulatory agent, an anti-angiogenic agent, and combinations thereof.

In some embodiments, an engineered cell of the present disclosure or composition thereof is administered with a steroid. The administration of a steroid can prevent or mitigate the risk of a subject receiving the engineered cell(s) or composition thereof having an autoimmune reaction.

The additional therapeutic agent may be administered by any suitable means. In some embodiments, the engineered cells described herein, populations thereof, or compositions thereof and the additional therapeutic agent is administered in the same pharmaceutical composition, e.g. by infusion. In some embodiments, the engineered cells described herein and additional therapeutic agent are included in different pharmaceutical compositions.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration. Generally, modes of administration include, without limitation, intravitreal, subretinal, suprachoroidal, intraarterial, intradermal, intramuscular, intraperitoneal, intravenous, nasal, parenteral, topical, pulmonary, and subcutaneous routes.

In embodiments where the engineered cells provided herein and the additional therapeutic agent are included in different pharmaceutical compositions, administration of the engineered cells provided herein can occur prior to, simultaneously, and/or following, administration of the additional therapeutic agent.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s)

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements/claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the statements/claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Example 1: Identification of Safe Harbour Sites

### Methods

### Short-Listing of Putative Safe Harbour Genomic Regions

A series of computational, whole-genome loci filtering criteria were applied to pick a narrow list of high-confidence, putative safe harbour sites for experimental validation. In the first step, genomic regions were selected that satisfy simultaneously all of the following criteria: the loci should be located outside of ultra-conserved regions (coordinates lifted over from hg 19 to hg38 assembly), outside of DNase clusters+/−2 kb (ENCFF503GCK, ENCODE database https://www.encodeproject.org), more than 50 kb away from any transcription start site and outside a gene transcription unit (ENSEMBL Release 103, dataset hsapiens_gene_ensembl, http://www.ensembl.org/index.html), more than 300 kb away from cancer-related genes (Cancer Gene Census, GRCh38, COSMIC v92 database https://cancer.sanger.ac.uk/census), more than 300 kb away from any miRNA (ENSEMBL Release 103, dataset hsapiens_gene_ensembl, gene_biotype=miRNA, http://www.ensembl.org/index.html) and more than 100 kb away from any long non-coding RNA (ENSEMBL Release 103, dataset hsapiens_gene_ensembl, gene_biotype=lncRNA, http://www.ensembl.org/index.html). From the filtered loci, those with high BLAT similarity to other sequences were discarded. On the RNA-seq level, the putative safe harbour sites had the requirement that they were associated with ubiquitously expressed, low variance genes. On the 3D chromosome organization level, the putative safe harbour sites had the requirement that they should belong to regions consistently located in active chromosomal compartments across multiple tissue types.

### Ubiquitously Expressed and Low-Variance Genes

From the median gene-level TPMs by tissue type (GTEx database, (https://www.gtexportal.org/home/datasets), an empirical set of low-variance housekeeping genes were identified. To this extent, the mean and the variance of each gene across all available tissue types were estimated and, independently, genes with the lowest, insignificant variability were selected using the HVG function of scran R package that decomposes the total variance of each gene into its biological and technical components. Genes whose expression levels do not change significantly across the tissue types (FDR>0.9) were picked and fitted tp a mean vs variance non-parametric lowers regression model. Genes were picked that had a mean TPM ≥5 and a variance below the average (smoothed) variance estimated from the lowest model.

### Loci Interaction Via Chromatin Conformation Capture

Using a set of publicly available Hi-C chromatin organization data from human cell and tissue types (Schmitt et al., Cell Rep. 17, 2042-2059, 2016), genomic regions consistently located (at least 20 out of 21 interrogated tissue types) in active (open chromatin) compartments were shortlisted.

### BLAT Analysis

The uniqueness of target loci was measured using BLAT (https://genome.ucsc.edu/cgi-bin/hgBlat) on the human genome GRCh38 with BLAT'S guess query type. BLAT takes the target DNA sequence as input and identifies similar ones in the whole human genome. Target sequences of more than 25,000 bps (BLAT's limit) were split into multiple smaller overlapping segments of length between 9,000 to 11,000 bps each (depending on the original target length) and tested separately. The location and length of the matching sequences, their similarity to the target (BLAT score) and the number of the matching base pairs were reported. Target loci with at least one matching sequence of more than 50% similarity were filtered out.

### TAD Boundary Check

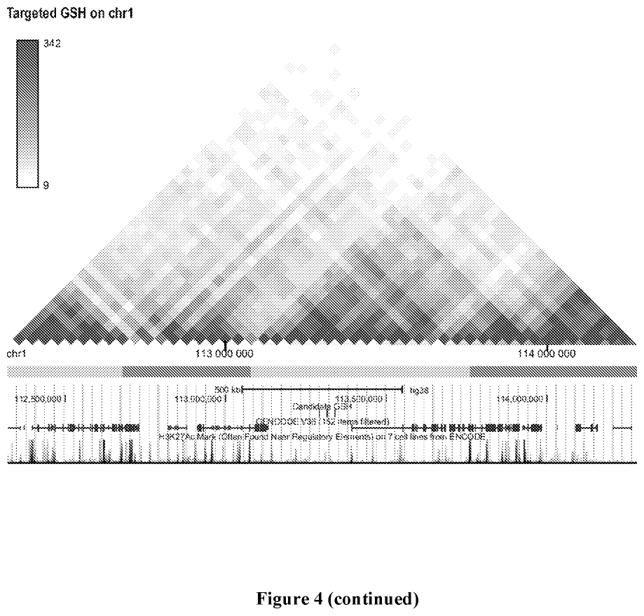

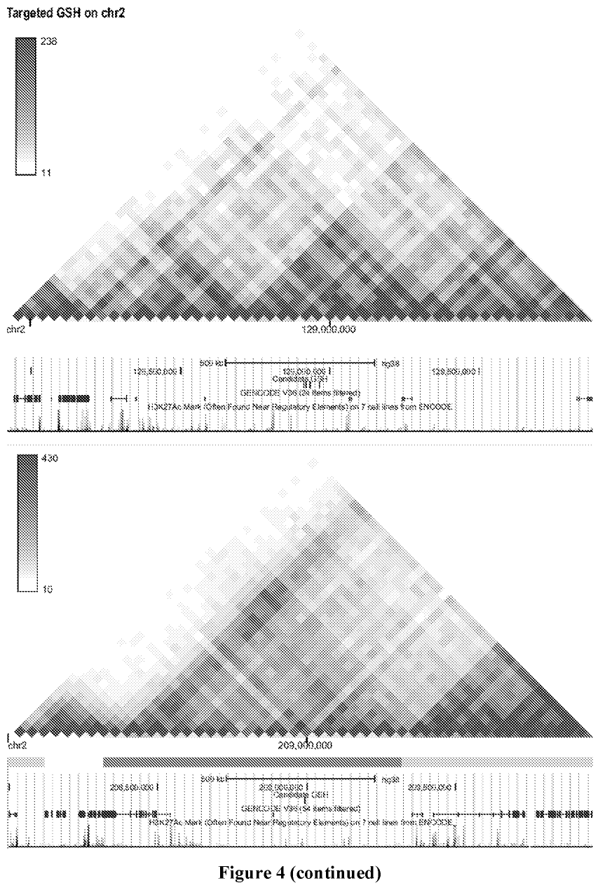

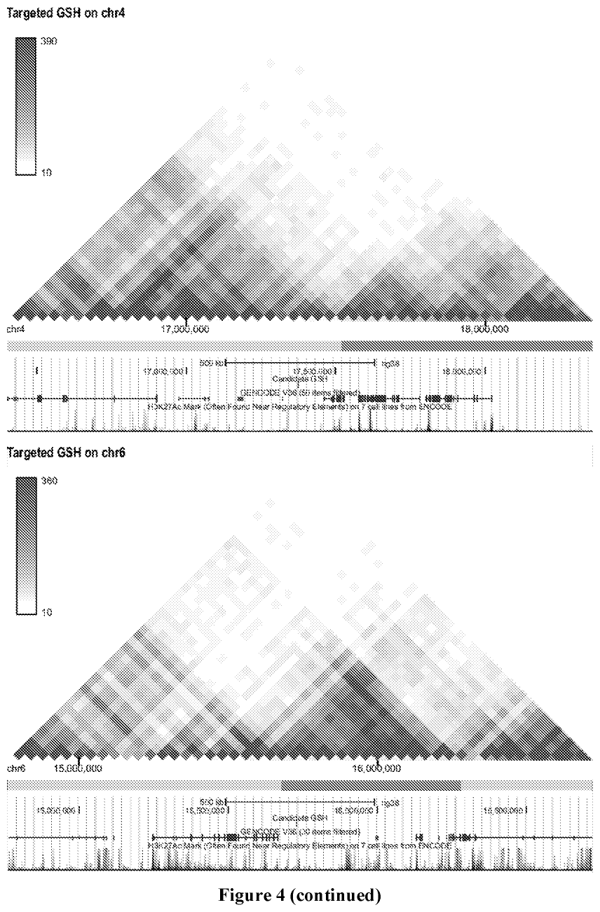

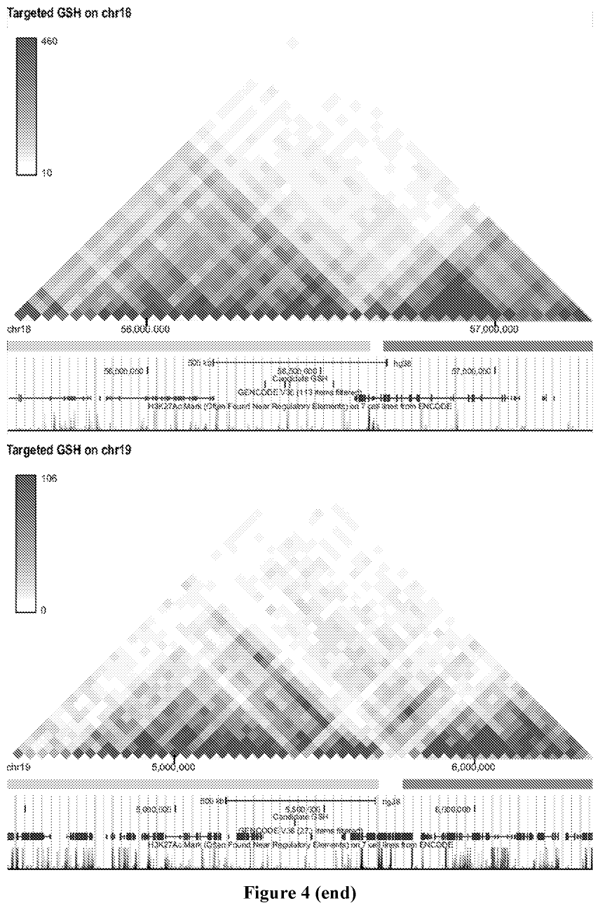

The locations of the in vitro targeted GSH candidates were checked against the TAD borders using data from H1 human embryonic stem cells (hESCs) on the 3D Genome Browser (http://3dgenome.org). Visual inspection of the candidate loci confirmed that all the candidate GSH are more than 80,000 bp away from TAD borders (FIG. 4).

### Results

### Computational Filtering for Safe and Accessible Loci

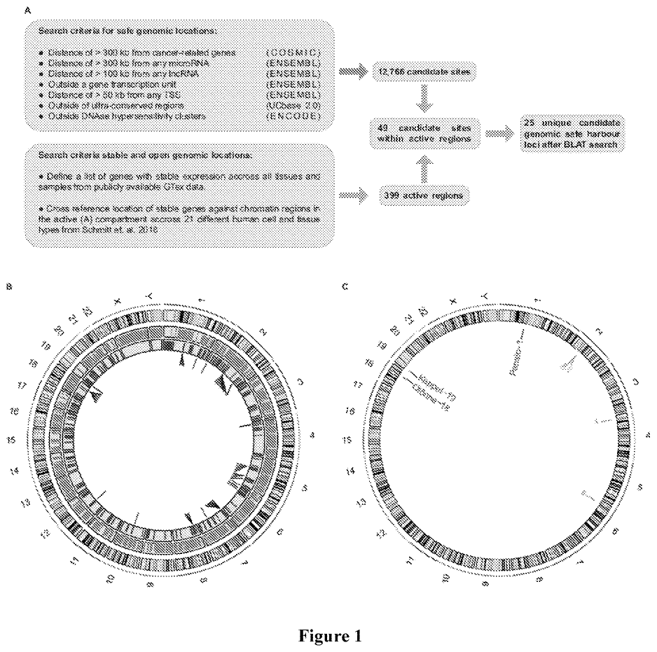

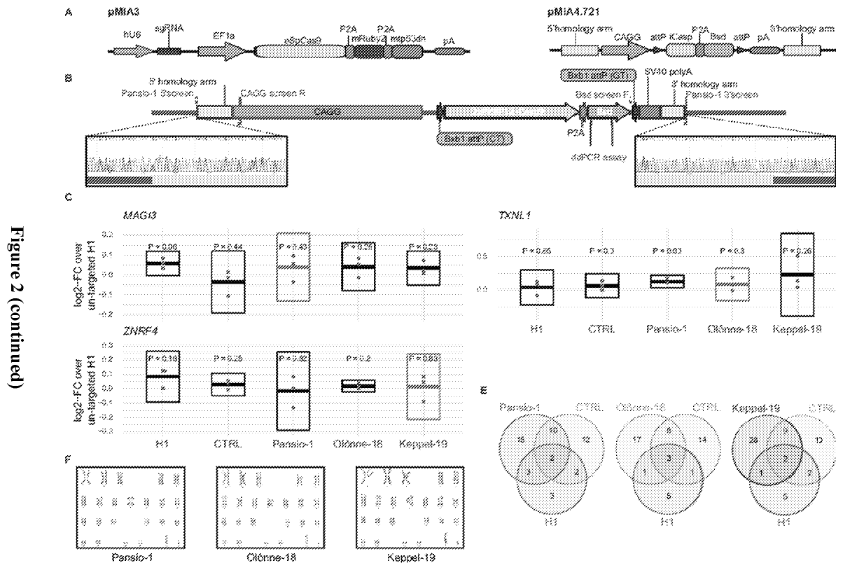

To shortlist putative genomic safe harbor sites (GSH), a computational search of the human genome was conducted using publicly available data (FIG. 1A) to identify targeting regions that lie outside DNAse clusters, gene transcription units and ultra-conserved regions, as well as being >300 kb away from any known oncogenes and micro RNAs (miRNAs), and >100 kb away from long non-coding RNAs (lncRNAs).

A safety criteria was included and a further filter was added to exclude any regions of DNaseI hypersensitivity, as these regions are likely enriched in transcription factor binding and regulatory elements. A total of 12,766 sites, ranging from 1 b to approximately 30 Mb, passed the filters used (FIG. 1A, 1B). For a universal GSH site to be useful, it not only needs to be safe, but also needs to enable stable expression of a transgene in any tissue type. The human genome was filtered for regions consistently in the active chromatin compartment based on 21 different human cell and tissue types. To extend the analysis beyond the limited set of samples, RNA-seq data of all available tissue types from the GTEx portal was utilized. An empirical set of ubiquitously expressed genes was selected with low variance. The chromosomal locations of these genes were cross-referenced with the consistently active chromatin regions. This analysis yielded 399 1 Mb active regions that overlapped with a ubiquitously expressed gene (FIG. 1A-B). By overlapping the two datasets, 49 safe sites were found within the active regions. The 49 sites were further filtered (FIG. 1A-B) using BLAT to generate a final shortlist of 25 unique putative universal GSH sites in the human genome (FIG. 1A-B, Table 1).

### Example 2: Validation of Safe Harbor Sites

### Methods

### Plasmid Construction

All restriction enzymes were purchased from NEB. PCR reactions were conducted using Q5® Hot Start High-Fidelity 2× Master Mix (NEB, M0494L). Ligations were conducted using isothermal assembly with NEBuilder® HiFi DNA Assembly Master Mix (NEB, E2621L). Primers used for fragment amplification are listed in Table 3.

pMIA4.721: An in-house expression plasmid containing a CAGG promoter (pMIA4.9) was digested with BamHI & SphI. Two gBlocks (bxb-bsd and bxb-sv) were directly ligated into the digested plasmid. The resulting plasmid was digested with PmlI & KpnI. A fragment containing codon optimised iCasp9-2A-Bsd was amplified from a plasmid supplied by Genewiz (sequence of iCasp9 based on Straathof et. al., Blood 105, 4247-4254, 2005) and ligated into the digested plasmid. This plasmid was subsequently digested with AgeI & SbfI. The SV40 polyA signal was amplified from pMAX-GFP and ligated to the digested plasmid to generate pMIA4.271.

To generate the HDR donors for each GSH candidate, the pMIA4.721 plasmid was digested with NheI for 5′ homology arm and with SbfI for 3′ homology arm. Homology arms ranging from 240 bp to 769 bp were amplified from H1 hESC genomic DNA. Ligation of homology arms was done in two sequential reactions. Order of ligation depended on the underlying sequence of homology arms for each target. Plasmid for control targeting was built by ligating 5′ homology arm to pMIA4.9 digested by NheI and 3′ by SbfI.

pMIA22: pMAX-GFP (Lonza) was digested with KpnI and SacI. A gBlock encoding a codon optimised BxbI-integrase with a C-terminal bi-partite nuclear localisation signal was amplified and ligated to the digested backbone.

pMIA10.5-Clover: An empty donor plasmid (pMIA10.5) containing a 5′ BxbI attB (CT) and a 3′BxbI attB (GT) was ordered from Genewiz. Clover transgene was amplified and ligated into the plasmid after digestion with AgeI & KpnI.

### Stem Cell Culture

Human ESC line H1 was maintained using mTeSR medium (Stemcell Technologies, 85850) on 1:200 Geltrex (Thermo fisher, A1413202) coated tissue culture plates and passaged regularly as cell aggregates every 4-5 days using ReLeSR (Stemcell Technologies, 05872).

CRISPR/Cas9 Mediated Targeted Construct Integration in hESC

H1 hESCs were targeted via nucleofection using an Amaxa-4D (Lonza). Briefly, gRNAs were designed using CRISPOR (http://crispor.tefor.net/). Three gRNAs with the highest predicted off-target scores and containing a native G-base in the first position were selected for each GSH candidate (Table 2). The gRNAs were cloned into pMIA3 plasmid (Addgene #109399) digested with Esp3I and tested via a GFP reconstitution assay in HEK239T cells. The target locus of each candidate GSH was amplified from H1 hESC gDNA (for primers see Table 4). The amplified target sequences, ranging from 232-974 bp were cloned into the pCAG-EGxxFP plasmid (Addgene #50716). The pMIA3 with the tested gRNA and the respective pCAG-EGxxFP target plasmid were transfected into HEK293T using lipofectamine3000 (ThermoFisher Scientific, L3000015), according to manufacturer's recommendations. For each candidate GSH the gRNA with the highest GFP signal at 48 h post transfection was selected for use in hESC targeting.

Five micrograms of pMIA3 plasmid containing optimal gRNA for each candidate GSH and the respective pMIA4.721 HDR-donor plasmids were nucleofected into hESC using the P3 Primary Cell kit (V4XP-3024) and programme CA-137. 1.5×10⁶ cells were used for each targeting and were plated onto geltrex coated wells on 6-well plates in mTeSR with CloneR (Stemcell Technologies, 05889) following nucleofection. After 24 h media was changed to mTeSR, and cells were allowed to recover for another 24-48 h. Once cells reached 70-80% confluency, Blasticidin (ThermoFisher Scientific, A1113903) was added to the culture media at 10 μg/ml. Individual colonies were manually picked from the wells after 7-14 days of selection and expanded further for screening.

### Junction PCR

Genomic DNA samples for all the collected GSH clones was isolated using PureLink™ Genomic DNA Mini Kit (ThermoFisher Scientific, K182002) according to manufacturer's instructions. PCR reactions amplifying both 5′ and 3′ targeting HDR junctions as well as the wild type allele were set up using primers listed in Table 4. Samples were checked for the correct amplification size and alignment of the Sanger sequencing reads for each junction PCR and wild type allele.

### Off-Target Analysis

The top five predicted off-targets were checked via PCR amplification and Sanger sequencing. PCR primers for respective off-targets for each gRNA are listed in Table 5.

### Copy Number Analysis

Blasticidin and RPP30 Copy Numbers were evaluated using Droplet Digital™ Polymerase Chain Reaction (ddPCR) technology (Bio-Rad Technologies), according to manufacturer's specifications. Briefly and following fluorescence-based quantification (ThermoFisher Scientific, Qubit™), 2.5 ng double-stranded DNA was added to a reaction mix containing target-specific primers/probe mixes (900 nM primer/250 nM probe per FAM and HEX fluorophore; Bio-Rad, 10042958 Unique Assay ID: dCNS626289650 and 10031243 Unique Assay ID: dHsaCP2500350), 0.05 U HaeIII Restriction Enzyme (New England Biolabs, R0108S) and ddPCR-specific Supermix for Probes (no dUTP) (Bio-Rad, 1863024). This was randomly partitioned into at least 10,000 discrete oil droplets per reaction using microfluidics within the QX200™ Droplet Generator (Bio-Rad, 1864002; together with Droplet Generation Oil for Probes, 1863005), which were gently transferred using a multi-channel pipette into a semi-skirted 96-well plate before heat-sealing (Bio-Rad PX1™ PCR Heat Sealer, 1814000). Target amplification within each droplet was conducted in the C1000 Touch™ Thermal Cycler with 96-Deep Well Reaction Module (Bio-Rad, 1851197) through the following PCR protocol: 1) Enzyme Activation at 95° C. for 10 minutes, 2) 40 cycles of Denaturation and annealing/extension at 94° C. for 30 seconds and 55° C. for 1 minute, respectively, 3) Enzyme Deactivation at 98° C. for 10 minutes. The QX200 Droplet Reader (Bio-Rad, 1864003) then derived the number of target-containing droplets through assessing each droplet for elevated, target-specific fluorescence. Blasticidin (FAM)-positive droplet counts were normalised using its respective well-specific RPP30 (HEX)-positive counts prior to downstream analysis. All experiments were done in duplicates, with data visualised and assessed using the QuantaSoft software version 1.7.4.917 (Bio-Rad).

qPCR Analysis

RNA was extracted from three biological replicates of the GSH targeted H1 hESC Pansio-1, Olônne-18 and Keppel-19, control targeted cells (cells that underwent CRISPR/Cas9-mediated targeting and HDR of an expression cassette at a non-GSH locus) and two independent cultures of untargeted cells using Direct-Zol™ RNA Miniprep kit (Zymo Research, ZYR.R2052). 1 μg of RNA was converted into cDNA with Superscript IV Vilo MM (ThermoFisher Scientific, 11766050). Quantitative PCR reactions using TaqMan gene expression assays and master mix were used to compare the expression levels of MAGI3, TXNL1 and ZNRF4 against reference genes 18S and GAPDH (ThermoFisher Scientific, Hs00326365_m1; Hs00169455_m1; Hs00741333_s1; HS99999901_S1; Hs03929097_g1 and 4444557). Quantitative RT-PCR analysis was done as described previously and resulting log 2 fold change gene expression data was compared against reference H1 untargeted samples.

### RNA-Seq Library Prep

RNA samples described above for qPCR were also used for RNA-seq. RNA concentration and quality were checked with an Agilent 2100 RNA Pico Chip (Agilent, 5067-1513). RNA sequencing libraries were prepared using the TruSeq Stranded Total RNA Sample Prep Kit (Illumina, 20020596) including Ribo-Zero to remove abundant cytoplasmic rRNA. The remaining intact RNA was fragmented, followed by first- and second-strand cDNA synthesis using random hexamer primers. “End-repaired” fragments were ligated with a unique illumina adapters. All samples were multiplexed and pooled into a single library. Sequencing was done on a HiSeq 4000 to a minimum depth of 50 million 150 bp paired-end reads per biological sample.

### RNA-Seq Quality Control

In all experiments, the raw paired-end reads in fastq format were initially processed with FastQC (https://www.bioinformatics.babraham.ac.uk/projects/fastqc/) for quality control at the base and sequence level. To remove the PCR duplicates, the FastUniq algorithm was utilised. The adaptor trimming was performed by Trimmomatic (version 0.39). The 229,649 annotated human transcripts of GENCODE v35 were quantified using Kallisto (version 0.46) followed by conversion of transcript to raw and TPM-normalized gene counts by the tximport package in R (Soneson et al., version 2, F1000Research 4, 1521, 2015). In total, 40,198 genes were quantified. Subsequently, QC was performed at the raw gene counts, checking for bad quality samples having less than 100,000 reads or more than 10% reads mapped to mitochondrial RNA or less than 2,000 detected genes. All samples of the various experiments were of high quality and were retained for the main analysis.

### RNA-Seq Differential Expression Analysis

The differential expression analysis was conducted by DEseq2 evaluating all pairwise comparisons of the conditions of each experiment. In each comparison, only the expressed genes, i.e. those with non-zero raw counts in at least one sample, were considered. The differentially expressed genes were those with |log FC|≥1 and FDR≤0.01.

### Functional Enrichment Analysis

Functional enrichment analysis was performed on the differentially expressed genes using the g: GOSt R package for g: Profiler (version e104_eg51_p15_3922dba) with g: SCS multiple testing correction method applying significance threshold of 0.05.

### Karyotyping

For each cell line, 20 GTL-banded metaphases were counted, of which a minimum of four have been analysed and karyotyped.

### Flow Cytometry

H1 wild type hESCs and Pansio-1, Olônne-18, and Keppel-19 H1 lines carrying Clover-transgene were disassociated with accutase (Stemcell Technologies, 07922) and resuspended in PBS. The single cells in PBS were analysed with a BD LSR Fortessa x-20 FACS Analyzer and FlowJo (v10.6.1).

hESC Cardiac Differentiation

Two days prior to starting differentiation, cells were dissociated using Accutase and seeded as single cells in Geltrex-coated 12-well plates at seeding density between 1 to 1.5×10⁶ cells. Cardiac differentiation was performed following the published protocol by Lian et. al. (Lian et al., Nat. Protoc. 8, 162-175, 2013), with modifications as follows. 6 μM of CHIR99021 (Stemcell Technologies, 72054) was added on day 0 and left for 24 h followed by medium change. On day 3, 5 μM IWP2 (Sigma Aldrich, 10536) was added using 50/50 mix of new fresh medium and conditioned medium collected from each well and left for 48 h. Culture medium from day 0 until day 7 was RPMI1640 (HyClone, SH30027.01) plus B-27 serum-free supplement without insulin (Gibco, A1895601). From day 7 and onwards RPMI1640 with B-27 serum free supplement with insulin (Gibco, 17504044) was used and changed every 2-3 days.

hESC Differentiation to Hepatocyte-Like Cells

hESCs were differentiated into hepatocyte-like cells as described previously (Hannan et al., Nat. Protoc. 8, 430-437, 2013; Ng et al., iScience 16, 192-205, 2019), with some modifications. Briefly, hESCs were dissociated into small clumps using ReLeSR and plated onto gelatin-coated coverslips in a 12-well plate with mTeSR. Two days later, hESCs were induced to differentiate into definitive endoderm (DE) cells in RPMI-1640 medium (Gibco) containing 2% B-27 (Invitrogen), 1% non-essential amino acids (Gibco), 1% GlutaMAX™ (Gibco) and 50 μM 2-mercaptoethanol (Gibco) (basal differentiation medium), supplemented with 100 ng/ml Activin A (R&D Systems), 3 μM CHIR99021 (Tocris) and 10 μM LY294002 (LC Labs) for the first three days (D0 to D3). From D3 to D6, cells were incubated in basal differentiation medium supplemented with 50 ng/ml Activin A to form foregut endoderm cells. From D6 to D10, cells were incubated in basal differentiation medium supplemented with 20 ng/ml BMP4 (Miltenyi Biotec) and 10 ng/ml FGF10 (Miltenyi Biotec) to form hepatic endoderm cells. From D10 to D24, hepatic endoderm cells were incubated in HCM Bulletkit (Lonza) differentiation media supplemented with 30 ng/ml Oncostatin M (Miltenyi Biotec) and 50 ng/ml HGF (Miltenyi Biotec). Differentiation medium was replaced every two or three days.

hESC Neural Induction

H1 cells cultured in mTeSR complete medium for 1-2 days were then used for neural induction as published (Li et al., Proc. Natl. Acad. Sci. U.S.A 108, 8299-8304, 2011; Wang et al., Genome Biol. 19, 1-12, 2017). Briefly, 20-30% confluent H1 cells were treated with CHIR99021, SB431542 and Compound E in neural induction media, changed every 2 days; 7 days later, the cells were split 1:3 by Accutase and seeded on matrigel-coated plates. ROCK inhibitor (1254, Tocris) was added (final concentration 10 μM) to the suspension at passaging. Cells were then cultured in neural cell culture medium. These derived cells are neural precursor cells (NPC), which were used for further studies.

### Neuronal Differentiation

Spontaneous neuronal differentiation was performed as previously described. Briefly, the derived 2×105 NPCs were seeded on poly-l-lysine (P4707, Sigma) and laminin (L2020, Sigma) coated 6-well plates in neural cell culture medium. The next day, the cells were cultured in neuron differentiation medium: DMEM/F12 (11330-032), Neurobasal (21103-049), 1×N2 (17502-048), 1×B27 (17504-044), 300 ng/ml cAMP (A9501), 0.2 mM vitamin C (A4544-25), 10 ng/ml BDNF (450-02), 10 ng/ml GDNF (450-10) until day 30.

### Immunofluorescence

Cells on coverslips were fixed in 4% paraformaldehyde (Wako) for 15 min at room temperature, before blocking in 5% donkey serum (EMD milipore) in PBS with 0.1% Triton X-100 for 1 h at room temperature. Cells were stained with primary antibody overnight at 4° C. (see Table 6 for antibodies used), or for control slides with blocking buffer. Secondary antibody staining was done with the appropriate AlexaFluor 594 for 1 h at room temperature. Lastly, cells were stained with DAPI (Sigma-Aldrich, 1:5000) for 20 min at room temperature. Coverslips were mounted onto glass slides using Vectashield (Vector Laboratories). Images were taken using the EVOS M5000 microscope. Light intensity and gain were kept consistent across samples and controls with each antibody.

### Results

Targeted Knock-In at Putative GSH with CRISPR/Cas9

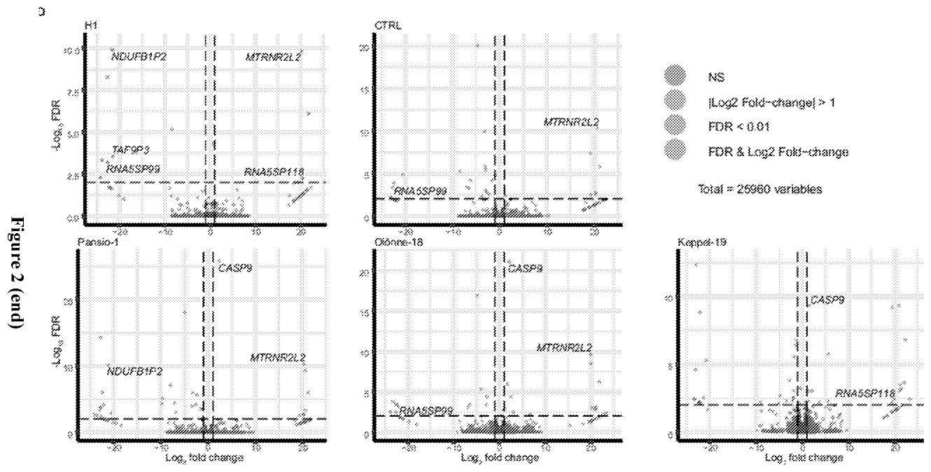

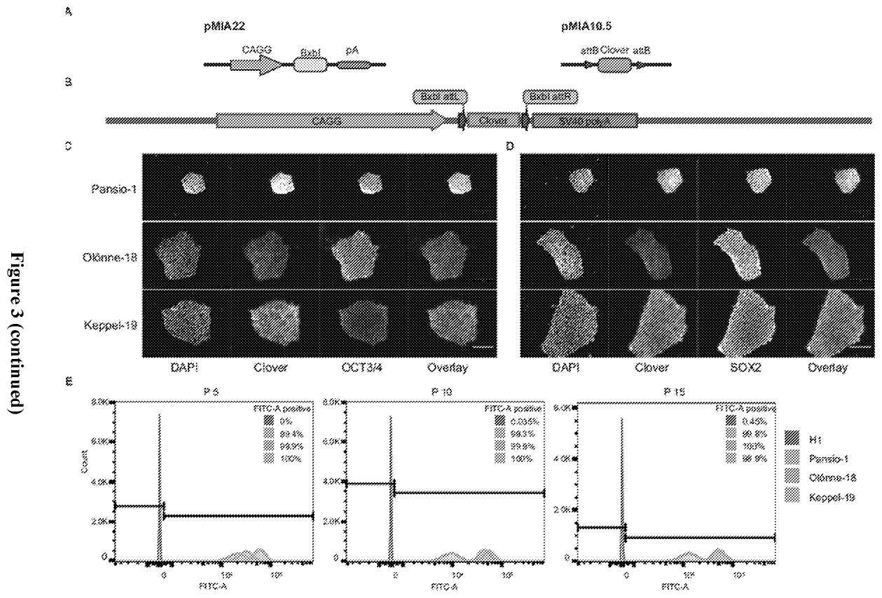

To validate the candidate GSHs, seven of the 25 sites were selected for in vitro experiments (FIG. 1C). None of the selected seven sites lie at or immediately adjacent to borders of topologically associated domains (TADs) (FIG. 4). H1 hESC was targeted using CRISPR/Cas9 and a donor landing pad construct (FIG. 2A) at each of the seven candidate sites. To exclude potential off-target effects of CRISPR/Cas9 targeting, a version of Cas9 with enhanced specificity and effective guides with highest predicted specificity available were used.

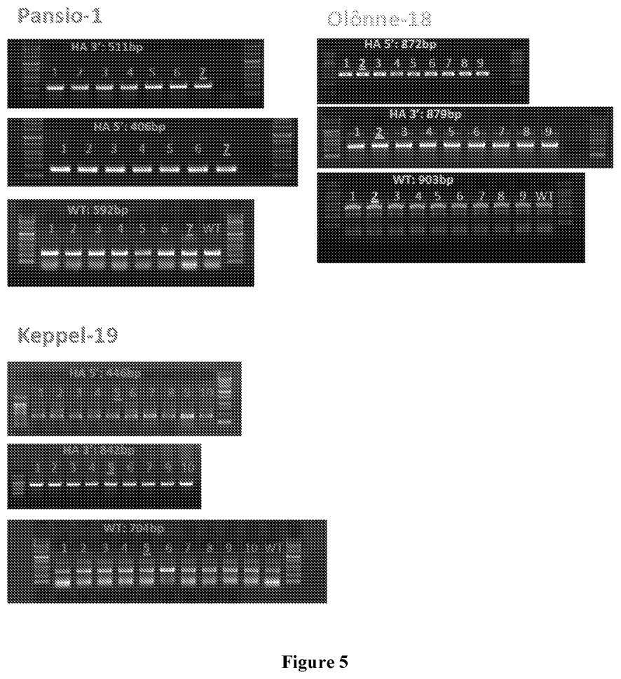

Following antibiotic selection, single clones were expanded and screened for successful homology directed repair driven integration of the expression construct with junction- and digital-PCR (FIG. 2B, Table 7, and FIG. 5). Successful heterozygous targeting of the donor construct was confirmed at three candidate GSH sites on chromosomes 1, 18 and 19. No evidence of off-target activity was observed following PCR amplification and Sanger sequencing of the top 5 predicted off-target sites for each of the targeted clones. The successfully targeted safe harbours were named after real world harbours, designating them Pansio-1, Olônne-18, and Keppel-19.

In Vitro Validation of Targeted GSH in hESCs

To investigate the safety of the targeted GSH, the mRNA expression levels of the nearest genes MAGI3, TXNL1 and ZNRF4 to Pansio-1, Olônne-18 and Keppel-19 respectively were checked using qPCR. When compared to un-targeted H1 hESCs, the 95% confidence intervals of log 2 fold-change (log 2-FC) for each gene in the respective GSH line overlapped with zero, indicating that the data do not show evidence of statistically significant change in mRNA expression levels of the nearest genes (FIG. 2C). RNA-seq analysis was conducted to look for gene expression changes on a global scale. The GSH targeted clones yielded very low numbers of differentially expressed (DE) genes; 30, 29 and 40 respectively for Pansio-1, Olônne-18, and Keppel-19 (FIG. 2D). Notably CASP9, the suicide gene included in the targeting construct, was the gene with lowest false discovery rate (FDR) in Pansio-1 and Olônne-18 and second lowest in Keppel-19 (FIG. 2D). A high proportion of the DE genes found in the GSH targeted lines were shared with control targeted H1 cells and/or untargeted wild-type H1 cells (FIG. 2E), suggesting that the observed changes were unrelated to the GSH targeting and transgene expression. The closest DE gene found from the analysis lies >42 Mb away for Pansio-1 and >47 Mb for Keppel-19. For Olônne-18 there were no DE genes on the same chromosome. Functional enrichment analysis of the DE genes revealed relatively few terms; 9, 8 and 15 for Pansio-1, Olônne-18, and Keppel-19 respectively. In the case of Pansio-1 all the terms were shared with the control targeted H1 cells except for one, which was due to the increased level of CASP9. As a further safety check, karyotyping of the three targeted clones was conducted and no abnormalities were observed (FIG. 2F). Taken together, these results suggest minimal disruption of the native genome and no karyotypic abnormalities following transgene integration to the three GSH.

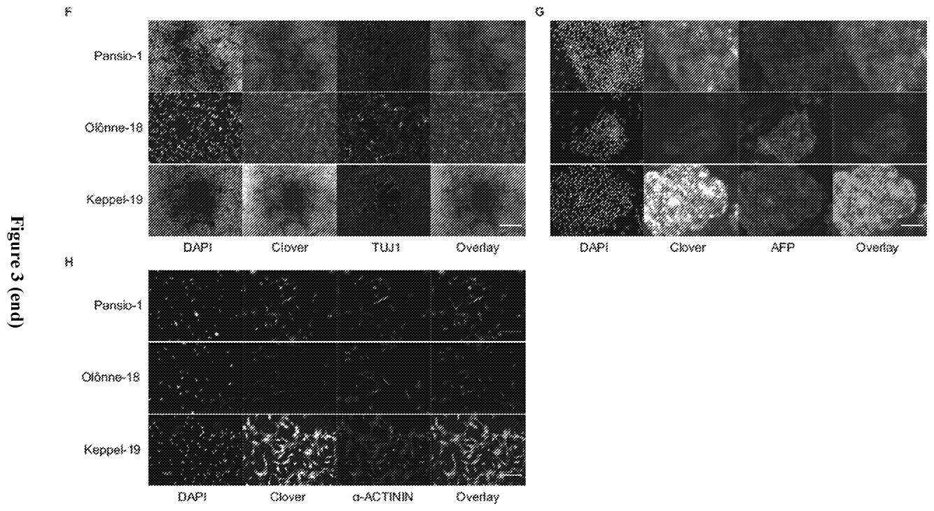

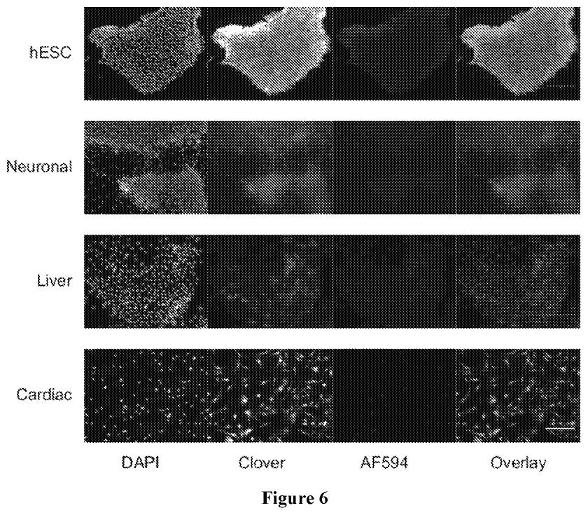

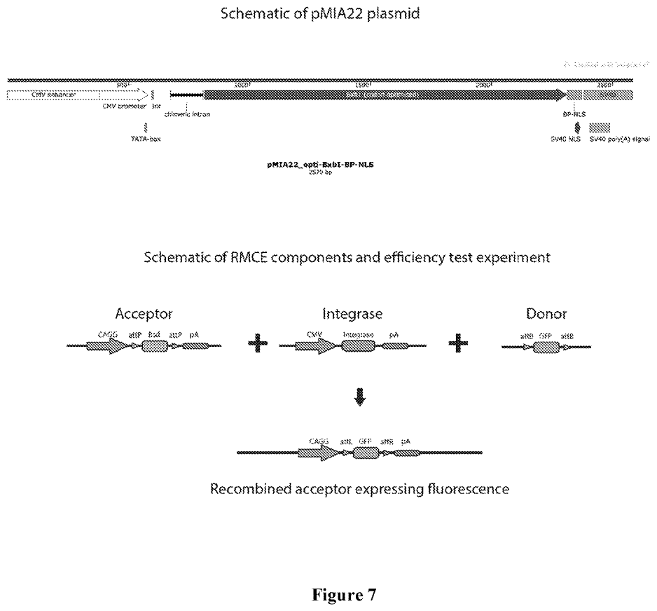

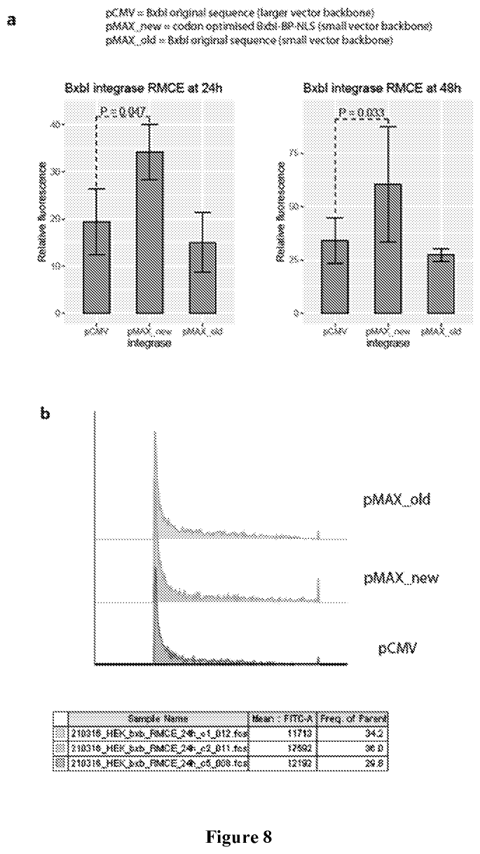

In addition to safety, a functional GSH also needs to allow for stable expression of a transgene. A sequence coding for Clover-fluorophore was swapped into the landing-pad design of the targeting construct by introducing a plasmid expressing BxbI-integrase as well as a donor construct into the three GSH lines (FIG. 3A-B). Targeted cells were enriched with fluorescence activated cell sorting. Introduction of the payload transgene did not alter the expression of pluripotency markers OCT3/4 and SOX2 (FIG. 3C-D). The Clover targeted GSH lines were maintained in hESC state over 15 passages and consistently observed >98% Clover positive cells (FIG. 3E). The levels of Clover integrated to Olônne-18 seemed to be consistently lower than integrations to Pansio-1 or Keppel-19 (FIG. 3C-E). To investigate the stability of the GSH in other cell types, directed differentiation of the Clover-integrated hESC lines into cell types from the three germ lineages was performed. Clover expression remained consistent in neuronal, liver, and cardiac cells (FIG. 3F-H, FIG. 6) in Pansio-1, Olônne-18, and Keppel-19 targeted cells.

Overall, a computational pipeline was developed to define GSH candidate sites from the human genome that fulfil criteria for safety as well as accessibility for transgene expression. The pipeline defines 25 unique candidate GSH and in vitro validation experiments were conducted for three of them, Pansio-1, Olônne-18, and Keppel-19. Targeting and transgene expression in hESC at the three sites led to minimal or no change in the expression levels of the nearest native genes or the transcriptome overall and did not interfere with directed differentiation to the three germ lineages. Furthermore, landing pad expression lines were established in H1 hESC of Pansio-1, Olônne-18, and Keppel-19.

### Discussion

A GSH site is an ideal location for transgene integration. To qualify as a GSH, a locus should be able to host transgenes enabling their stable expression as well as not interfere with the native genome. A number of previous studies have reported discovery and usage of a handful of integration sites, which fulfil a subset of criteria previously suggested for GSH. However, in contrast to the candidate sites presented herein (FIG. 1A-B), the previously reported sites did not utilize criteria to avoid potential regulatory elements or criteria for universally stable and active genomic regions. Directed differentiation of the Pansio-1, Olônne-18 and Keppel-19 targeted hESC showed consistent expression in hESC and cells from all three germ lineages

(FIG. 3C-H), whereas majority of the previously reported sites remain studied in only a limited number of cell types.

The three candidate sites tested herein, Pansio-1, Olônne-18, and Keppel-19, demonstrated stable expression of the Clover transgene in both hepatocyte- and cardiomyocyte-like cells after differentiation from hESC (FIG. 3G-H). The transcriptome analysis of the GSH targeted hESC lines showed very low number of differentially expressed genes (FIG. 2D) when compared to untargeted H1 hESC. Furthermore, many of the DE genes observed were shared with independent wild type H1 hESC as well as control targeted H1 hESC (FIG. 2E). The nearest observed DE genes to each targeted candidate GSH were located >42 Mb away from the targeted site, which is far beyond the distance generally suggested for enhancer-promoter interactions and TADs.

The three candidate GSH, Pansio-1, Olônne-18, and Keppel-19 are able to support stable transgene expression in different cell types, and integration at these sites shows minimal perturbation of the native genome. As hESC currently offer the best available model to test the stability of transgene expression from a GSH in the human genome, landing-pad H1 hESC lines were generated for all three candidate sites. These cell lines will allow easy integration of various transgenes to the candidate GSH for research applications.

TABLE 1
Coordinates of the 25 genomic safe harbor sites, their associated active chromosome
regions & housekeeping gene, and BLAT score against the most similar region.

| | | | | Active | Active | | |
|---|---|---|---|---|---|---|---|
| | | | | region | region | Housekeeping | |
| Chromosome | Start | End | Width | start | end | gene | BLAT-score |
| 1 | 113289036 | 113289342 | 307 | 113000001 | 114000000 | HIPK1 | 0.21 |
| 1 | 113314841 | 113318369 | 3529 | 113000001 | 114000000 | HIPK1 | 0.18 |
| 1 * | 113339961 | 113340514 | 554 | 113000001 | 114000000 | HIPK1 | 0.27 |
| 2 * | 128912721 | 128914814 | 2094 | 128000001 | 129000000 | UGGT1 | 0.08 |
| 2 | 128918961 | 128919839 | 879 | 128000001 | 129000000 | UGGT1 | 0.16 |
| 2 * | 128932307 | 128935799 | 3493 | 128000001 | 129000000 | UGGT1 | 0.15 |
| 2 | 128963272 | 128965759 | 2488 | 128000001 | 129000000 | UGGT1 | 0.44 |
| 2 | 208992998 | 208997459 | 4462 | 208000001 | 209000000 | PIKFYVE | 0.28 |
| 4 * | 17373361 | 17374159 | 799 | 17000001 | 18000000 | MED28 | 0.04 |
| 5 | 131058585 | 131058947 | 363 | 131000001 | 132000000 | FNIP1 | 0.41 |
| 5 | 148753741 | 148757219 | 3479 | 148000001 | 149000000 | FBXO38 | 0.10 |
| 6 * | 15727241 | 15727490 | 250 | 15000001 | 16000000 | DTNBP1 | 0.47 |
| 7 | 4314741 | 4315279 | 539 | 4000001 | 5000000 | FOXK1 | 0.15 |
| 7 | 4321017 | 4323839 | 2823 | 4000001 | 5000000 | FOXK1 | 0.09 |
| 7 | 4328040 | 4329659 | 1620 | 4000001 | 5000000 | FOXK1 | 0.21 |
| 7 | 4353504 | 4354219 | 716 | 4000001 | 5000000 | FOXK1 | 0.32 |
| 7 | 4454808 | 4456201 | 1394 | 4000001 | 5000000 | FOXK1 | 0.17 |
| 8 | 23945241 | 23945819 | 579 | 23000001 | 24000000 | R3HCC1 | 0.34 |
| 8 | 23986981 | 23988319 | 1339 | 23000001 | 24000000 | R3HCC1 | 0.06 |
| 8 | 23999628 | 24001194 | 1567 | 23000001 | 24000000 | R3HCC1 | 0.02 |
| 18 | 56339813 | 56340245 | 433 | 56000001 | 57000000 | TXNL1 | 0.06 |
| 18 | 56396821 | 56397319 | 499 | 56000001 | 57000000 | TXNL1 | 0.07 |
| 18 | 56410681 | 56411039 | 359 | 56000001 | 57000000 | TXNL1 | 0.14 |
| 18 * | 56534775 | 56536439 | 1665 | 56000001 | 57000000 | TXNL1 | 0.16 |
| 19 * | 5400761 | 5402139 | 1379 | 5000001 | 6000000 | SAFB | 0.18 |

* = GSH shortlisted for in vitro validation

TABLE 2
gRNAs used for genome targeting. Preferred gRNAs are shaded.

| SEQ ID No. | Target | sgRNA ID | sgRNA sequence |
|---|---|---|---|
| 1 | Ts1 | 511 rev | GGUCACUUCAGACCUGGUAU |
| 2 | | 430 rev | 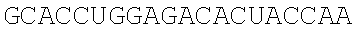 |
| 3 | | 490 rev | GGAGUCUCCCCAAAGCUCUG |
| 4 | Ts2a | 610 forw | 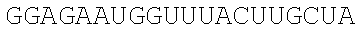 |
| 5 | | 151 rev | GGACUCCUUUAACAAAUCCA |
| 6 | | 154 forw | GGAGUAAUUAUUGUUGACCU |
| 7 | Ts2c | 774 rev | GGAAUUGAACCAGUGUGUUG |
| 8 | | 260 rev | GAAGAAGUAGCAAGCAUGUG |
| 9 | | 810 rev | 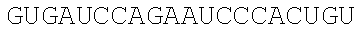 |
| 10 | Ts4 | 449 forw | 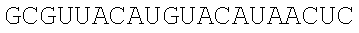 |
| 11 | | 23 forw | GGUGUCUAUCUGCUAAUAAC |
| 12 | | 24 forw | GUGUCUAUCUGCUAAUAACA |
| 13 | Ts6 | 163 forw | GGGGAUCAGUUGGGUCUGCC |
| 14 | | 143 forw | 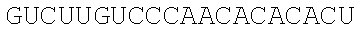 |
| 15 | | 119 rev | GUGUGUGUUGGGACAAGACU |
| 16 | Ts18 | 1023 rev | GGUGAUUAGCUCUCGCUAUG |
| 17 | | 961 rev | 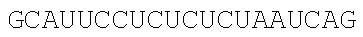 |
| 18 | | 460 rev | GACUGCAGGUGCAUAAAGAG |
| 19 | Ts19 | 141 rev | 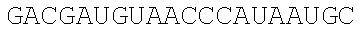 |
| 20 | | 629 forw | GUGGGCACAGGUAUGCGUGC |
| 21 | | 142 forw | GACAAGGGUCUCUCAUUGGC |

TABLE 3
Primers used for plasmid construction

| SEQ | | | |
|---|---|---|---|
| ID No. | Plasmid | Name | Primer sequence (5′-3′) |
| 22 | pMIA4.721 | iCasp9 into 4.7_fwd | tgtacggtacaaacccacacgtgGCCACCATGC |
| | | | TCGAGGGA |
| 23 | | iCasp9 into 4.7_rev | gaccagacaaaccacggtaccTTATTAGCCCT |
| | | | CCCACACATAACC |
| 24 | | sv40 into 4.72_fwd | ggtgtacggtacaaacccaaccggGTTTGGACA |
| | | | AACCACAAC |
| 25 | | sv40 into 4.72_rev | gccaagcttgcatgcctgcaggtTACCACATTTG |
| | | | TAGAGGTTTTAC |
| 26 | | Ts1 5′ into | atcgcccttcccaacagttgGCCAGTTGCAAA |
| | | 4.721_fwd | AAGAGAATC |
| 27 | | Ts1 5′ into 4.721_rev | aatgtcgatcgacgatgatgCAAAGGCATAAT |
| | | | CCGTAATG |
| 28 | | Ts1 3′ into | tacaaatgtggtaacctgcaGTAGTGTCTCCAG |
| | | 4.721_fwd | GTGCAAG |
| 29 | | Ts1 3′ into 4.721_rev | gccaagcttgcatgcctgcaGTGTATTTAGAT |
| | | | ACAGTTAGATAAAGTCATG |
| 30 | | Ts2a 5′ into | gatcgcccttcccaacagttGGGCTAAGTTATA |
| | | 4.721_fwd | AAACTCTTG |
| 31 | | Ts2a 5′ into | caatgtcgatcgacgatgatCAAGTAAACCAT |
| | | 4.721_rev | TCTCCATTC |
| 32 | | Ts2a 3′ into | aacctctacaaatgtggtaaCTATGGAAAACA |
| | | 4.721_fwd | ATTTGGC |
| 33 | | Ts2a 3′ into | gccagtgccaagcttgcatgCCATAAAGCTTT |
| | | 4.721_rev | TGAGATTC |
| 34 | | Ts2c 5′ into | atcgcccttcccaacagttgCTAGAGTCACCAT |
| | | 4.721_fwd | GCTGC |
| 35 | | Ts2c 5′ into | aatgtcgatcgacgatgatgTGTTGGGCATATA |
| | | 4.721_rev | TCCAAAG |
| 36 | | Ts2c 3′ into | aacctctacaaatgtggtaaTGGGATTCTGGAT |
| | | 4.721_fwd | CACATTTG |
| 37 | | Ts2c 3′ into | gccagtgccaagcttgcatgGCAGAAGAATGA |
| | | 4.721_rev | CATCAGATC |
| 38 | | Ts4 5′ into | gatcgcccttcccaacagttTGTCTATCTGCTA |
| | | 4.721_fwd | ATAACAG |
| 39 | | Ts4 5′ into 4.721_rev | caatgtcgatcgacgatgatTTATGTACATGTA |
| | | | ACGCTG |
| 40 | | Ts4 3′ into | aacctctacaaatgtggtaaCTCTGGGTGAGTA |
| | | 4.721_fwd | TACAAC |
| 41 | | Ts4 3′ into 4.721_rev | gccagtgccaagcttgcatgACTAAAGGGAGA |
| | | | TGACTTAC |
| 42 | | Ts6 5′ into | gatcgcccttcccaacagttTCCTCACATCCCA |
| | | 4.721_fwd | GACGATG |
| 43 | | Ts6 5′ into 4.721_rev | caatgtcgatcgacgatgatGTGTGTTGGGAC |
| | | | AAGACTAG |
| 44 | | Ts6 3′ into | aacctctacaaatgtggtaaACTGGGGATCAGT |
| | | 4.721_fwd | TGGGTC |
| 45 | | Ts6 3′ into 4.721_rev | gccagtgccaagcttgcatgGCTAGGCTATGT |
| | | | TTTTGACCC |
| 46 | | Ts18 5′ into | gatcgcccttcccaacagttTCAATACTGCATG |
| | | 4.721_fwd | AGTTCCGC |
| 47 | | Ts18 5′ into | caatgtcgatcgacgatgatTCAGAGGCAGAG |
| | | 4.721_rev | TGGGGAAATAG |
| 48 | | Ts18 3′ into | aacctctacaaatgtggtaaTTAGAGAGAGGA |
| | | 4.721_fwd | ATGCTTC |
| 49 | | Ts18 3′ into | gccagtgccaagcttgcatgAGGTAACCTTTT |
| | | 4.721_rev | CAGATTC |
| 50 | | Ts19 5′ into | gatcgcccttcccaacagttAGGAGCCAGAAG |
| | | 4.721_fwd | TGCAAAG |
| 51 | | Ts19 5′ into | caatgtcgatcgacgatgatATGCCGGCCAAT |
| | | 4.721_rev | GAGAGAC |
| 52 | | Ts19 3′ into | aacctctacaaatgtggtaaTATGGGTTACATC |
| | | 4.721_fwd | GTCTC |
| 53 | | Ts19 3′ into | gccagtgccaagcttgcatgCATATTCACACA |
| | | 4.721_rev | GCATTCTC |
| 54 | pMIA22 | new-bxb into | tttgcctttctctccacaggccaccATGCGGGCAC |
| | | pMAX_fwd | TCGTTGTAATC |
| 55 | | new-bxb into | gtggtttgtccaaactcatcCTATTATTCGACCT |
| | | pMAX_rev | TCCTCTTCTTCTTC |
| 56 | pMIA10.5- | clover into 10.5_fwd | ccgtcgtcaggatcatccggCCACCATGGTGA |
| | Clover | | GCAAGG |
| 57 | | clover into 10.5_rev | gtcgacaagccggccggtactaTTACTTGTACA |
| | | | GCTCGTCCATG |
| 58 | pCAG- | Ts1 into | aggatccgctagcctgcaggCATTGTGCTAAG |
| | EGxxFP | eGxxFP_fwd | TGAAAC |
| 59 | | Ts1 into | gcttgccgatatcgaattcgGGAAAGCAAATT |
| | | eGxxFP_rev | GCTGTTG |
| 60 | | Ts2a into | aggatccgctagcctgcaggGCAACTGTATAT |
| | | eGxxFP_fwd | CCACATTC |
| 61 | | Ts2a into | gcttgccgatatcgaattcgGTGACCTTTTGTG |
| | | eGxxFP_rev | TCTGAC |
| 62 | | Ts2c into | aggatccgctagcctgcaggGGAATACATTGT |
| | | eGxxFP_fwd | CATGTTATG |
| 63 | | Ts2c into | gcttgccgatatcgaattcgGTATGCTGTTGGT |
| | | eGxxFP_rev | GATAATG |
| 64 | | Ts4 into | aggatccgctagcctgcaggAGCAAAGTAATA |
| | | eGxxFP_fwd | CTCTCCTG |
| 65 | | Ts4 into | gcttgccgatatcgaattcgTTCATAATATGAA |
| | | eGxxFP_rev | ACTCTAAGGTG |
| 66 | | Ts6 into | aggatccgctagcctgcaggAGGGAGGTTGCA |
| | | eGxxFP_fwd | GTGAGC |
| 67 | | Ts6 into | gcttgccgatatcgaattcgAAGTCACTCCCAA |
| | | eGxxFP_rev | CTGGG |
| 68 | | Ts18 into eGxx_fwd | aggatccgctagcctgcaggTCAATACTGCAT |
| | | | GAGTTCCGC |
| 69 | | Ts18 into eGxx_rev | gcttgccgatatcgaattcgAGGCATCAGGCTG |
| | | | GTGAG |
| 70 | | Ts19 into | aggatccgctagcctgcaggAGGAGCCAGAA |
| | | eGxxFP_fwd | GTGCAAAG |
| 71 | | Ts19 into | gcttgccgatatcgaattcgGATCACAGGCAG |
| | | eGxxFP_rev | GAGCCAC |
| 72 | pMIA4.9 | Tc11 5′ into 4.9_fwd | GATCGCCCTTCCCAACAGTTGCAGA |
| | | | ATCACACAGTCCATTTTG |
| 73 | | Tc11 5′ into 4.9_rev | CAATGTCGATCGACGATGATTCACC |
| | | | GGTAACAGGTGCTG |
| 74 | | Tc11 3′ into 4.9_fwd | gtatcttatcatgtctgcggTTTGTGTTCCCCCA |
| | | | GAAAG |
| 75 | | Tc11 3′ into 4.9_rev | gccaagcttgcatgcctgcaCTGAAGCTGGAG |
| | | | CAACATG |

TABLE 4
Primers used for junction PCR and WT allele screening.

| SEQ | | | | Expected |
|---|---|---|---|---|
| ID | | | Primer sequence | amplicon |
| No. | Screen | Name | (5′-3′) | size |
| 76 | Junction | Ts1 5′ screen F | CATTGTGCTAAGTGAAAC | 406 |
| 77 | PCR | Ts2a 5′ screen F | GCAACTGTATATCCACATTC | 676 |
| 78 | | Ts2c 5′ screen F | GGAATACATTGTCATGTTATG | 705 |
| 79 | | Ts4 5′ screen F | AGCAAAGTAATACTCTCCTG | 639 |
| 80 | | Ts6 5′ screen F | GGAAGAGGCGCTCCT | 689 |
| 81 | | Ts18 5′ screen F | TCCATCTGCAAATGACAATATG | 872 |
| 82 | | Ts19 5′ screen F | CTTGAGCCCAGGAAGTC | 446 |
| 83 | | CAGG screen R | ATGGGCTATGAACTAATGAC | — |
| 84 | | Bsd screen F | TATGTGTGGGAGGGCTAA | — |
| 85 | | Ts1 3′ screen R | GGAAAGCAAATTGCTGTTG | 297 |
| 86 | | Ts2a 3′ screen R | GTGACCTTTTGTGTCTGAC | 404 |
| 87 | | Ts2c 3′ screen R | TCAAAATACATTAGAAAACTAT | 904 |
| | | | ACTAATCAAAACAC | |
| 88 | | Ts4 3′ screen R | GAGACCTCAGAACTCATCTAGC | 430 |
| 89 | | Ts6 3′ screen R | AGGAGTTAGGTGTGCCA | 925 |
| 90 | | Ts18 3′ screen R | TGATTCAATATATGTTGATTAAT | 627 |
| | | | TTTGC | |
| 91 | | Ts19 3′ screen R | GCTGCTGCTTCTTGAGG | 842 |
| 92 | WT | Ts1 WTF | CATTGTGCTAAGTGAAAC | 592 |
| 93 | allele | Ts1 WTR | GGAAAGCAAATTGCTGTTG | |
| 94 | | Ts2a WTF | GCAACTGTATATCCACATTC | 974 |
| 95 | | Ts2a WT R | GTGACCTTTTGTGTCTGAC | |
| 96 | | Ts2c WTF | GGAATACATTGTCATGTTATG | 777 |
| 97 | | Ts2c WT R | GTATGCTGTTGGTGATAATG | |
| 98 | | Ts4 WTF | AGCAAAGTAATACTCTCCTG | 684 |
| 99 | | Ts4 WTR | TTCATAATATGAAACTCTAAGG | |
| | | | TG | |
| 100 | | Ts6 WTF | AGGGAGGTTGCAGTGAGC | 232 |
| 101 | | Ts6 WTR | AAGTCACTCCCAACTGGG | |
| 102 | | Ts18 WTF | TCAATACTGCATGAGTTCCGC | 903 |
| 103 | | Ts18 WTR | AGGCATCAGGCTGGTGAG | |
| 104 | | Ts19 WTF | AGGAGCCAGAAGTGCAAAG | 848 |
| 105 | | Ts19 WTR | GATCACAGGCAGGAGCCAC | |

TABLE 5
Primers used for predicted off-target screening.

| SEQ | | | Primer sequence | Expected | Predicted off- |
|---|---|---|---|---|---|
| ID No. | GSH | Name | (5′-3′) | amplicon size | target site |
| 106 | Pansio-1 | 1430top1 fwd | TGCCAGTGGTGA | 267 | chr17: |
| | | | TGTACT | | 60769199- |
| 107 | | 1430top1 rev | ACAGCTACCTCC | | 60769221 |
| | | | CTACAC | | |
| 108 | | 1430top2-II fwd | TTAtttCTGTGTAA | 383 | chr5: |
| | | | ACTTTCA | | 158353919- |
| 109 | | 1430top2-II rev | aacCTGAAGTATC | | 158353941 |
| | | | TTGTG | | |
| 110 | | 1430top3 fwd | GTACCTAACACA | 271 | chr10: |
| | | | TAACTGG | | 60052591- |
| 111 | | 1430top3 rev | CAGTAAATACTC | | 60052613 |
| | | | TTCCCC | | |
| 112 | | 1430top4-II fwd | TCCACTGAGAAT | 258 | chr3: |
| | | | GACCCT | | 81380212- |
| 113 | | 1430top4-II rev | CAACCTTATACT | | 81380234 |
| | | | CTGGGA | | |
| 114 | | 1430top5 fwd | ACTGGAAAATGG | 406 | chr15: |
| | | | GTAGAC | | 31241962- |
| 115 | | 1430top5 rev | CCATGGGGTAGG | | 31241984 |
| | | | TTGATT | | |
| 116 | Olonne-18 | 18961top 1 fwd | ACCACAAGAACT | 480 | chr3: |
| | | | CAAACT | | 39624207- |
| 117 | | 18961top 1 rev | CCCCTCTCATTTT | | 39624229 |
| | | | TTGTCTA | | |
| 118 | | 18961top2 fwd | GAAGAATATGCA | 370 | chrX: |
| | | | TAGGGAG | | 78836657- |
| 119 | | 18961top2 rev | AGGTGAATAGGA | | 78836679 |
| | | | GATTGC | | |
| 120 | | 18961top3 fwd | GGTGCCCTTTGA | 269 | chr13: |
| | | | AGATTT | | 53754357- |
| 121 | | 18961top3 rev | TGGGATCCTCAA | | 53754379 |
| | | | GACATT | | |
| 122 | | 18961top4 fwd | AGCCATTTAGTC | 273 | chr2: |
| | | | AGTACAG | | 19382456- |
| 123 | | 18961top4 rev | TACTATGAGAGT | | 19382478 |
| | | | GTTGTGAG | | |
| 124 | | 18961top5 fwd | GTGTCAAGGGGA | 302 | chrX: |
| | | | TAATCT | | 55415052- |
| 125 | | 18961top5 rev | TTGTGTATGGAC | | 55415074 |
| | | | AGGAGGA | | |
| 126 | Keppel-19 | 19141top 1-II | CAGATTCTGACA | 209 | chr2: |
| | | fwd | GAAGG | | 143930861- |
| 127 | | 19141top 1-II | ATACTAAAGACA | | 143930883 |
| | | rev | GAGAATGTTA | | |
| 128 | | 19141top2 fwd | TACCCAAAGGCT | 265 | chr2: |
| | | | CTAACC | | 30119441- |
| 129 | | 19141top2 rev | ACATGATCCTCC | | 30119463 |
| | | | TCTCTT | | |
| 130 | | 19141top3 fwd | GACCAAGAAAC | 409 | chr3: |
| | | | AATAGACC | | 161328139- |
| 131 | | 19141top3 rev | GCTTGTCTATTC | | 161328161 |
| | | | CTCTTG | | |
| 132 | | 19141top4 fwd | AAGAGAATGGG | 432 | chr14: |
| | | | ACATAGC | | 60775546- |
| 133 | | 19141top4 rev | TCTCACCTCAAT | | 60775568 |
| | | | CATAGC | | |
| 134 | | 19141top5 fwd | CAGGTTTTTTCCT | 438 | chr3: |
| | | | TCAGCA | | 135735796- |
| 135 | | 19141top5 rev | TTAATCATGGCA | | 135735818 |
| | | | GCAGGG | | |

TABLE 6
Antibodies used for immunofluorescence staining.

| Antibody | Antigen | Supplier | Catalogue # | Dilution used |
|---|---|---|---|---|
| Primary | OCT3/4 | Santa-Cruz | sc-9081 | 1:100 |
| Primary | SOX2 | Abcam | ab97959 | 1:250 |
| Primary | TUJ1 | Covance | MRB-435P | 1:1000 |
| Primary | AFP | Sigma | A8452 | 1:100 |
| Primary | α-ACTININ | Sigma | A7811 | 1:500 |
| Secondary | Mouse | Invitrogen | A-11005 | 1:500 |
| Secondary | Rabbit | Invitrogen | A-11012 | 1:500 |
| Secondary | Goat | Invitrogen | A-11058 | 1:500 |

Sequence of optimised BxbI
(SEQ ID NO: 136)
ATGCGGGCACTCGTTGTAATCAGGTTGTCTCGAGTTACGGATGCG
ACCACGTCCCCGGAGAGACAACTCGAATCATGCCAGCAGCTTTGT
GCTCAGAGAGGATGGGACGTTGTGGGCGTGGCCGAAGATCTCGAC
GTATCAGGGGCGGTGGACCCCTTCGATAGAAAGAGAAGACCAAAT
CTTGCCAGGTGGCTGGCTTTCGAAGAGCAACCTTTTGACGTAATC
GTGGCGTATAGAGTAGACAGGCTCACGAGGTCTATCCGGCACCTT
CAACAATTGGTTCACTGGGCAGAAGACCACAAGAAGTTGGTGGTT
TCAGCAACTGAGGCGCACTTCGATACTACCACTCCATTTGCGGCA
GTCGTGATTGCCCTTATGGGTACGGTTGCCCAAATGGAACTGGAG
GCTATTAAGGAGCGGAATAGGAGTGCCGCACATTTTAATATCCGG
GCTGGCAAATATCGCGGATCACTCCCCCCGTGGGGTTACCTCCCT
ACCCGCGTAGACGGTGAGTGGAGGCTCGTACCGGACCCCGTTCAG
AGAGAGCGGATCTTGGAAGTGTATCACAGAGTAGTGGACAACCAT
GAACCTTTGCATCTCGTAGCGCATGATCTCAATCGCAGGGGGGTA
CTCTCTCCAAAAGATTATTTCGCGCAACTTCAGGGTCGGGAGCCC
CAGGGAAGAGAATGGTCTGCAACAGCTCTCAAGCGGAGTATGATA
AGTGAAGCCATGCTTGGATACGCCACTCTTAACGGGAAAACCGTC
AGAGACGACGATGGCGCACCACTCGTCAGGGCGGAGCCCATTTTG
ACACGGGAACAACTTGAGGCGCTTCGGGCAGAACTGGTTAAGACC
AGTCGCGCTAAGCCTGCAGTGTCTACTCCCTCCCTCTTGCTCAGA
GTCCTGTTTTGCGCAGTTTGTGGTGAGCCCGCCTATAAATTTGCT
GGAGGCGGACGGAAGCACCCAAGATACCGCTGCAGAAGCATGGGA
TTTCCCAAACACTGCGGGAATGGAACAGTCGCTATGGCCGAGTGG
GACGCCTTTTGCGAAGAGCAGGTGCTTGATTTGCTGGGAGACGCT
GAACGGCTGGAAAAAGTGTGGGTTGCAGGGTCAGATAGTGCCGTA
GAGTTGGCGGAGGTTAACGCAGAACTTGTGGATCTGACTTCTTTG
ATTGGCTCCCCCGCGTATCGGGCCGGAAGCCCCCAACGAGAAGCC
CTTGACGCCCGAATTGCGGCCCTCGCAGCCCGACAAGAGGAACTG
GAAGGACTTGAAGCCCGCCCTAGTGGATGGGAGTGGCGGGAGACA
GGACAAAGGTTTGGAGACTGGTGGAGAGAACAAGATACTGCGGCG
AAGAACACATGGCTGCGATCAATGAACGTCCGGCTGACGTTTGAC
GTTCGGGGCGGCTTGACAAGGACTATTGATTTCGGGGATCTGCAA
GAATATGAACAGCATCTTAGACTGGGCTCTGTAGTTGAAAGGCTT
CACACCGGTATGTCC
Sequence of optimised BxbI-BP-NLS
(SEQ ID NO: 137)
ATGCGGGCACTCGTTGTAATCAGGTTGTCTCGAGTTACGGATGCG
ACCACGTCCCCGGAGAGACAACTCGAATCATGCCAGCAGCTTTGT
GCTCAGAGAGGATGGGACGTTGTGGGCGTGGCCGAAGATCTCGAC
GTATCAGGGGCGGTGGACCCCTTCGATAGAAAGAGAAGACCAAAT
CTTGCCAGGTGGCTGGCTTTCGAAGAGCAACCTTTTGACGTAATC
GTGGCGTATAGAGTAGACAGGCTCACGAGGTCTATCCGGCACCTT
CAACAATTGGTTCACTGGGCAGAAGACCACAAGAAGTTGGTGGTT
TCAGCAACTGAGGCGCACTTCGATACTACCACTCCATTTGCGGCA
GTCGTGATTGCCCTTATGGGTACGGTTGCCCAAATGGAACTGGAG
GCTATTAAGGAGCGGAATAGGAGTGCCGCACATTTTAATATCCGG
GCTGGCAAATATCGCGGATCACTCCCCCCGTGGGGTTACCTCCCT
ACCCGCGTAGACGGTGAGTGGAGGCTCGTACCGGACCCCGTTCAG
AGAGAGCGGATCTTGGAAGTGTATCACAGAGTAGTGGACAACCAT
GAACCTTTGCATCTCGTAGCGCATGATCTCAATCGCAGGGGGGTA
CTCTCTCCAAAAGATTATTTCGCGCAACTTCAGGGTCGGGAGCCC
CAGGGAAGAGAATGGTCTGCAACAGCTCTCAAGCGGAGTATGATA
AGTGAAGCCATGCTTGGATACGCCACTCTTAACGGGAAAACCGTC
AGAGACGACGATGGCGCACCACTCGTCAGGGCGGAGCCCATTTTG
ACACGGGAACAACTTGAGGCGCTTCGGGCAGAACTGGTTAAGACC
AGTCGCGCTAAGCCTGCAGTGTCTACTCCCTCCCTCTTGCTCAGA
GTCCTGTTTTGCGCAGTTTGTGGTGAGCCCGCCTATAAATTTGCT
GGAGGCGGACGGAAGCACCCAAGATACCGCTGCAGAAGCATGGGA
TTTCCCAAACACTGCGGGAATGGAACAGTCGCTATGGCCGAGTGG
GACGCCTTTTGCGAAGAGCAGGTGCTTGATTTGCTGGGAGACGCT
GAACGGCTGGAAAAAGTGTGGGTTGCAGGGTCAGATAGTGCCGTA
GAGTTGGCGGAGGTTAACGCAGAACTTGTGGATCTGACTTCTTTG
ATTGGCTCCCCCGCGTATCGGGCCGGAAGCCCCCAACGAGAAGCC
CTTGACGCCCGAATTGCGGCCCTCGCAGCCCGACAAGAGGAACTG
GAAGGACTTGAAGCCCGCCCTAGTGGATGGGAGTGGCGGGAGACA
GGACAAAGGTTTGGAGACTGGTGGAGAGAACAAGATACTGCGGCG
AAGAACACATGGCTGCGATCAATGAACGTCCGGCTGACGTTTGAC
GTTCGGGGCGGCTTGACAAGGACTATTGATTTCGGGGATCTGCAA
GAATATGAACAGCATCTTAGACTGGGCTCTGTAGTTGAAAGGCTT
CACACCGGTATGTCCAAAAGGACCGCAGATGGCTCCGAGTTTGAG
TCACCGAAGAAGAAGAGGAAGGTCGAATAA
Sequence of GSH locus Chrl 1: 113339961-
113340514
(SEQ ID NO: 138)
GTGGATGAATGAATAAAGATGATGTGGTATGTACATACAATGGAA
TATTAGCCTTGGAAAAGAAGGAAATACTTTCACATGCTACAACAT
GGATGAACCTTGAGGACATTGTGCTAAGTGAAACAAGCCAGTTGC
AAAAAGAGAATCTGCTTGCATGGAGTATGTAAAAAAGTCATATTC
TTGGAAAGTAGAATGGACATTGCCAGGGGAGGAAGTGGGAAGTTC
AATGGATGCAGAGTTTTAGTTTTGCAAGATGAAAAGTTCCTAGGG
ATCTATTGCACAACAATGTGCATGTAGTTGACACTACTGAACTGT
GTATTGAAAAAGGGTTAAGATTGCAAAAATCAATCTTAGGCAATT
TGATTTTTCAAGACACAGACATCTACAAATGTTATTATTGAAGAA
ACCAATGCATCATTACGGATTATGCCTTTGGTAGTGTCTCCAGGT
GCAAGGAATAACAGAAATAAAAGTCACATGATGTGTGAACCCCAG
AGCTTTGGGGAGACTCCTATACCAGGTCTGAAGTGACCTAATAGG
AAGTAGCTGTTATT
Sequence of GSH locus Chrl 18: 56534775-
56536439
(SEQ ID NO: 139)
CCACAAAGTTTTGGGGTAAATTGTTGCACAGCAATAAATAACTCG
AATGAAAATATTCTGATTAAGTCTTTCTGATGCTAATTTGACTTT
GCTTCTTCCAAAAATTAGTCCAAATGAGTAAAATGATCATTTCCT
CTTGGCATGTCTATAGATTTTCTACTTGGCATCTTCTCCATCTGC
AAATGACAATATGCCTTTGGTTTTCTGGTATTCCATGGAAGCTAC
ATGTGTCAAGAAGTCAATACTGCATGAGTTCCGCAAATAAACCAT
CCCACTCACCATCACTGTTCATGCATTTCCTACAACAGCCTATAT
CTAGTTCATCCATGTCCGCTTCTATCTTTTCTCTCCCAGGTTTCT
GCAGCAATCATACTCTCATTTCTTTCTAGGGGCTCTAACTTCTGC
TTCTCTGCTTGTAGGCCTATGTCCTTCTCTTCTGTTAATGACATT
GGGCTTTTCCCCCTCTTTATGCACCTGCAGTCAAGGTGGGAAACA
AAAAGATTCGAAACTATTTTGACTCAGACCTAGGAACTTGTAATG
TCACCATAAGATGAGATTCCTGTCTCCAGCCAAATCTCACCTTGA
ATTGTAATAATCCCTATGTGTCAACGGAGGAGCCAGGTAGAGATA
ATTGAATTATAGGGGTGGTTTCCCCCATACTGTTCTCATGGTAGT
GAATAAGTCTCACAAGATCTGATGGTTCTATAAATGGAAGATCCC
CCGCACAAGCTCTCTTGCCTGCTACCATGTAAGACATGCCTTTGC
TTCTCCTTTACCTTCTGCCATGATTGTAAGGCCTCCCCAGCCAGG
TGGAATTGTGAGTCCATTAAGCCTCTTTTATATATATATAAATTA
CCCAGTCTCAGATATGTCTTTATTAACAGTGTGAGAACAGACTAC
AACCTGACCCTGACAATCCTTGTGCTTCCCTCATCTTGCCTCTTC
TATTTCCCCACTCTGCCTCTGATTAGAGAGAGGAATGCTTCTGGG
GTAACTGCTAATTGAAAGTAGGTTAGTCTCTTCCACATAGCGAGA
GCTAATCACCCATGATCCACAATTCCAGGAGCATGAACATGTATC
TCCCTCACCAGCCTGATGCCTGAAATTCAAATGCAAGAGATTCAT
TCCTTGAAATTAGATATTGGCTCTAAAGATTCTTTTAAAGTGGGA
GGTGGCCAGAATGAGATGATTGCTATAGTCAGGTTTAACAATCAA
CTGAAATACCCCAGAATCTGAAAAGGTTACCTCTAAGAAGGAAAA
CTAGTAAGTGAATTCTATCAACATGCAAAATTAATCAACATATAT
TGAATCATACTAGCTGCTTTGGTAAACATCTTATCCTAGAGTTTT
AATAGTGTTCTAAAAAGTTGTCTATAAAGGTGCACCCGAGGCTGG
GCACAATGGCTCCTGACTGTAATCCCAGCAATTTGGGAGGCCAGG
GAAGGTGGATAACTTGAGGCCAGGTGTTTGAGATCAGCCTGGGGA
ACACAGAGAAACCCCATCTCTACTAAAAAACAGAAAAATTAGCAG
GGTGCTGTGGCGCGCACCTGTAGTCCCAGCTACTCGGGAGGCTGA
GGCACAAGAATCGCTTGAGCCTGGGAGATGGAGGTTGCAGTGAAC
CGAGATCACGCCACTGCCCTCCAGCCTGGGCGACAGAGTGAGACG
Sequence of GSH locus Chrl 19: 5400761-5402139
(SEQ ID NO: 140)
ACCAAGGACCAGTGGTGGGAATGAGCTTGGAGACACGGACACACA
GAATCAGAGACAGACCGAAACAAAGAGACAGAGACAGGCGCTCAG
GGACACCCTAACAGAGACAGACAGACAAAGAGACAAGGGTCTCTC
ATTGGCCGGCATTATGGGTTACATCGTCTCTCCCCCAAAAAACCT
ATATGCTGAAGTCCTCACCCCTCAGAACCTCCGAATGTACTGGAG
GTGGTACAGATGTAACTGGAGTAGGGTGGGCTCTAATCTAATACG
ACTGATGACCTTATAAAAGGGGACAATGTGGTTCATGCCTGTAAT
CCCAGCACTTTGGGAGGCTGAGGCGGGGGGGATCACTTGAGGTCA
GGAGTTCGAGACCAGCCTGGCCAACATGGCAAAACCCTGTCTCTA
CTAAAAATAGAAAAATTAGCTGGGTGTGGTCGTGGGCGCCTGTAA
TCCAAGCTACTCCAGAGGCTGAGGCAGGAGAATCCCTTGAACCTG
GGAGGCGGAGGTTGCAGTGAGCTGAAATCGTGCCATTGCTCTCCA
GCCTGGGCAACAGAGTGAGAAGATTCCTTCTCAAAACAAACAAAA
AAACGAAGGGGCAGGGGGACAATGTGGGCACAGGTATGCGTGCAG
GGAGAATGCTGTGTGAATATGAAGTTGCCATCTACAAACCAAGGA
GAAAGGCCTGGGACAGACCCCTCCCTTACATCCCTCAAGAAGCAG
CAGCCGGCCAGGCATGGTGGCTCCTGCCTGTGATCCCAGTGCTTT
TCAGAGGCTGAGATGGGAGGATGCTTGAGGCCACGAGCTCAAGAC
CAGCCTGGGCAACAGAATGAGACCTCGTCTCTATTAAAAGTAAAA
AAAAAAAAAAAAAAAAAAAATACCCAAGTGTGGTGGCACAGGCTT
GTGGTCCCAGCTACTCAGGAAGGTGAGGTGGAGGATCACTTGAGC
CCACGAGGTCGAGGCTGCAGTGAGCTGTGATTGCATCATTGCACT
CCAGCCTGGGTGACAGAGCAAGACCTTGTCTCAGAAAACTGAAAG
AAAGGGAAGGAAGGAAGGAAGGAAGGGAGGGACGAAGGGAAGGAG
GGAGGGAGGGAAGGAGGAAGGAAGGAGGAAGAGAGGCAGGGAGGA
AGGGAAGGAAGGAGGGCAGGAAGAAGGAAGAGAGGGTTGGGAGGA
GGGAAGAAGGAAGAGGGGGGAGGGAGGCAGGGGGGAAGGAGGGAA
GGAAGGAAGGAAAGAAGGAAGGAAGGAAAGAAGGGAGGGAGGAAA
GGAAGGAGGAAGAGAAGGGAGGGAGGAAAGAAGGAAGGAAGGAAG
GAAAAAAGAACCAGCCCTGCTGGCAACTTGGTTTCAGACTTCTAG
CCTCCAGGACTGTGACATATTTCTGTTTT

1. An engineered cell comprising at least one heterologous polynucleotide inserted into a safe harbour locus listed in Table 1.

2. The engineered cell of claim 1, wherein the safe habour locus is selected from Chrl 1:113339961-113340514, Chrl 18:56534775-56536439 or Chrl 19:5400761-5402139.

3. The engineered cell of claim 2, wherein the integration site is Chrl 1: 113340395-113340396, Chrl 18:56535740-56535741 or Chrl 19:5400906-5400907.

4. The engineered cell of claim 1, wherein the engineered cell is a human cell.

5. The engineered cell of claim 1, wherein the engineered cell is a primary cell, a stem cell, an immune cell or a cell line.

6. The engineered cell of claim 5, wherein the stem cell is an embryonic stem cell (ESC) or an induced pluripotent stem cell (iPSC).

7. The engineered cell of claim 5, wherein the immune cell is a T cell or an NK cell.

8. The engineered cell of claim 1, wherein the heterologous polynucleotide comprises one or more site-specific recombination sequences.

9. The engineered cell of claim 8, wherein the site-specific recombination sequence is a recognition sequence recognized by a site-specific recombinase, optionally wherein the recognition sequence comprises an attP site or attB site that is recognized by a BxbI integrase.

10. (canceled)

11. The engineered cell of claim 1, wherein the heterologous polynucleotide comprises a transgene that is operably linked to a promoter.

12. The engineered cell of claim 11, wherein the transgene encodes a recombinant protein, optionally a therapeutic agent.

13. A composition comprising the engineered cell of claim 1 and a pharmaceutical excipient.

14. A method of editing a cell or preparing a master clonal cell line, the method comprising inserting at least one heterologous polynucleotide into a safe harbour locus in the cell, wherein the safe harbour locus is a safe harbour locus listed in Table 1.

15. The method of claim 14, wherein the at least one heterologous polynucleotide is inserted into the safe harbour locus using homology-directed repair or homology-independent targeted insertion.

16. (canceled)

17. The method of claim 14, wherein the heterologous polynucleotide comprises one or more site-specific recombination sequences.

18. The method of claim 17, wherein the site-specific recombination sequence is a recognition sequence recognized by a site-specific recombinase, optionally wherein the recognition sequence comprises an attP site or attB site that is recognized by a BxbI integrase.

19. (canceled)

20. The method of claim 14, wherein the heterologous polynucleotide comprises a transgene that is operably linked to a promoter.

21. The method of claim 20, wherein the transgene encodes a recombinant protein, optionally a therapeutic agent.

22. A method of editing a cell, the method comprising contacting the cell with one or more gRNA, at least one heterologous polynucleotide, and one or more Cas9 endonucleases, wherein the one or more gRNAs and Cas9 endonucleases facilitate the insertion of the at least one heterologous polynucleotide into chromosomal DNA within a safe harbor locus, wherein the safe harbor locus is a safe harbour locus listed in Table 1.

23-25. (canceled)


SEQUENCE LISTING (140 sequences)

SEQ ID NO: 1 - RNA, 20 nt, synthetic construct
  source 1..20: mol_type = other RNA
ggtcacttcagacctggtat

SEQ ID NO: 2 - RNA, 20 nt, synthetic construct
  source 1..20: mol_type = other RNA
gcacctggagacactaccaa

SEQ ID NO: 3 - RNA, 20 nt, synthetic construct
  source 1..20: mol_type = other RNA
ggagtctccccaaagctctg

SEQ ID NO: 4 - RNA, 20 nt, synthetic construct
  source 1..20: mol_type = other RNA
ggagaatggtttacttgcta

SEQ ID NO: 5 - RNA, 20 nt, synthetic construct
  source 1..20: mol_type = other RNA
ggactcctttaacaaatcca

SEQ ID NO: 6 - RNA, 20 nt, synthetic construct
  source 1..20: mol_type = other RNA
ggagtaattattgttgacct

SEQ ID NO: 7 - RNA, 20 nt, synthetic construct
  source 1..20: mol_type = other RNA
ggaattgaaccagtgtgttg

SEQ ID NO: 8 - RNA, 20 nt, synthetic construct
  source 1..20: mol_type = other RNA
gaagaagtagcaagcatgtg

SEQ ID NO: 9 - RNA, 20 nt, synthetic construct
  source 1..20: mol_type = other RNA
gtgatccagaatcccactgt

SEQ ID NO: 10 - RNA, 20 nt, synthetic construct
  source 1..20: mol_type = other RNA
gcgttacatgtacataactc

SEQ ID NO: 11 - RNA, 20 nt, synthetic construct
  source 1..20: mol_type = other RNA
ggtgtctatctgctaataac

SEQ ID NO: 12 - RNA, 20 nt, synthetic construct
  source 1..20: mol_type = other RNA
gtgtctatctgctaataaca

SEQ ID NO: 13 - RNA, 20 nt, synthetic construct
  source 1..20: mol_type = other RNA
ggggatcagttgggtctgcc

SEQ ID NO: 14 - RNA, 20 nt, synthetic construct
  source 1..20: mol_type = other RNA
gtcttgtcccaacacacact

SEQ ID NO: 15 - RNA, 20 nt, synthetic construct
  source 1..20: mol_type = other RNA
gtgtgtgttgggacaagact

SEQ ID NO: 16 - RNA, 20 nt, synthetic construct
  source 1..20: mol_type = other RNA
ggtgattagctctcgctatg

SEQ ID NO: 17 - RNA, 20 nt, synthetic construct
  source 1..20: mol_type = other RNA
gcattcctctctctaatcag

SEQ ID NO: 18 - RNA, 20 nt, synthetic construct
  source 1..20: mol_type = other RNA
gactgcaggtgcataaagag

SEQ ID NO: 19 - RNA, 20 nt, synthetic construct
  source 1..20: mol_type = other RNA
gacgatgtaacccataatgc

SEQ ID NO: 20 - RNA, 20 nt, synthetic construct
  source 1..20: mol_type = other RNA
gtgggcacaggtatgcgtgc

SEQ ID NO: 21 - RNA, 20 nt, synthetic construct
  source 1..20: mol_type = other RNA
gacaagggtctctcattggc

SEQ ID NO: 22 - DNA, 41 bp, synthetic construct
  source 1..41: mol_type = other DNA
tgtacggtacaaacccacacgtggccaccatgctcgaggga

SEQ ID NO: 23 - DNA, 45 bp, synthetic construct
  source 1..45: mol_type = other DNA
gaccagacaaaccacggtaccttattagccctcccacacataacc

SEQ ID NO: 24 - DNA, 43 bp, synthetic construct
  source 1..43: mol_type = other DNA
ggtgtacggtacaaacccaaccggtgtttggacaaaccacaac

SEQ ID NO: 25 - DNA, 46 bp, synthetic construct
  source 1..46: mol_type = other DNA
gccaagcttgcatgcctgcaggttaccacatttgtagaggttttac

SEQ ID NO: 26 - DNA, 41 bp, synthetic construct
  source 1..41: mol_type = other DNA
atcgcccttcccaacagttggccagttgcaaaaagagaatc

SEQ ID NO: 27 - DNA, 40 bp, synthetic construct
  source 1..40: mol_type = other DNA
aatgtcgatcgacgatgatgcaaaggcataatccgtaatg

SEQ ID NO: 28 - DNA, 40 bp, synthetic construct
  source 1..40: mol_type = other DNA
tacaaatgtggtaacctgcagtagtgtctccaggtgcaag

SEQ ID NO: 29 - DNA, 51 bp, synthetic construct
  source 1..51: mol_type = other DNA
gccaagcttgcatgcctgcagtgtatttagatacagttagataaagtcatg

SEQ ID NO: 30 - DNA, 42 bp, synthetic construct
  source 1..42: mol_type = other DNA
gatcgcccttcccaacagttgggctaagttataaaactcttg

SEQ ID NO: 31 - DNA, 41 bp, synthetic construct
  source 1..41: mol_type = other DNA
caatgtcgatcgacgatgatcaagtaaaccattctccattc

SEQ ID NO: 32 - DNA, 39 bp, synthetic construct
  source 1..39: mol_type = other DNA
aacctctacaaatgtggtaactatggaaaacaatttggc

SEQ ID NO: 33 - DNA, 40 bp, synthetic construct
  source 1..40: mol_type = other DNA
gccagtgccaagcttgcatgccataaagcttttgagattc

SEQ ID NO: 34 - DNA, 38 bp, synthetic construct
  source 1..38: mol_type = other DNA
atcgcccttcccaacagttgctagagtcaccatgctgc

SEQ ID NO: 35 - DNA, 40 bp, synthetic construct
  source 1..40: mol_type = other DNA
aatgtcgatcgacgatgatgtgttgggcatatatccaaag

SEQ ID NO: 36 - DNA, 41 bp, synthetic construct
  source 1..41: mol_type = other DNA
aacctctacaaatgtggtaatgggattctggatcacatttg

SEQ ID NO: 37 - DNA, 41 bp, synthetic construct
  source 1..41: mol_type = other DNA
gccagtgccaagcttgcatggcagaagaatgacatcagatc

SEQ ID NO: 38 - DNA, 40 bp, synthetic construct
  source 1..40: mol_type = other DNA
gatcgcccttcccaacagtttgtctatctgctaataacag

SEQ ID NO: 39 - DNA, 39 bp, synthetic construct
  source 1..39: mol_type = other DNA
caatgtcgatcgacgatgatttatgtacatgtaacgctg

SEQ ID NO: 40 - DNA, 39 bp, synthetic construct
  source 1..39: mol_type = other DNA
aacctctacaaatgtggtaactctgggtgagtatacaac

SEQ ID NO: 41 - DNA, 40 bp, synthetic construct
  source 1..40: mol_type = other DNA
gccagtgccaagcttgcatgactaaagggagatgacttac

SEQ ID NO: 42 - DNA, 40 bp, synthetic construct
  source 1..40: mol_type = other DNA
gatcgcccttcccaacagtttcctcacatcccagacgatg

SEQ ID NO: 43 - DNA, 40 bp, synthetic construct
  source 1..40: mol_type = other DNA
caatgtcgatcgacgatgatgtgtgttgggacaagactag

SEQ ID NO: 44 - DNA, 39 bp, synthetic construct
  source 1..39: mol_type = other DNA
aacctctacaaatgtggtaaactggggatcagttgggtc

SEQ ID NO: 45 - DNA, 41 bp, synthetic construct
  source 1..41: mol_type = other DNA
gccagtgccaagcttgcatggctaggctatgtttttgaccc

SEQ ID NO: 46 - DNA, 41 bp, synthetic construct
  source 1..41: mol_type = other DNA
gatcgcccttcccaacagtttcaatactgcatgagttccgc

SEQ ID NO: 47 - DNA, 43 bp, synthetic construct
  source 1..43: mol_type = other DNA
caatgtcgatcgacgatgattcagaggcagagtggggaaatag

SEQ ID NO: 48 - DNA, 39 bp, synthetic construct
  source 1..39: mol_type = other DNA
aacctctacaaatgtggtaattagagagaggaatgcttc

SEQ ID NO: 49 - DNA, 39 bp, synthetic construct
  source 1..39: mol_type = other DNA
gccagtgccaagcttgcatgaggtaaccttttcagattc

SEQ ID NO: 50 - DNA, 39 bp, synthetic construct
  source 1..39: mol_type = other DNA
gatcgcccttcccaacagttaggagccagaagtgcaaag

SEQ ID NO: 51 - DNA, 39 bp, synthetic construct
  source 1..39: mol_type = other DNA
caatgtcgatcgacgatgatatgccggccaatgagagac

SEQ ID NO: 52 - DNA, 38 bp, synthetic construct
  source 1..38: mol_type = other DNA
aacctctacaaatgtggtaatatgggttacatcgtctc

SEQ ID NO: 53 - DNA, 40 bp, synthetic construct
  source 1..40: mol_type = other DNA
gccagtgccaagcttgcatgcatattcacacagcattctc

SEQ ID NO: 54 - DNA, 46 bp, synthetic construct
  source 1..46: mol_type = other DNA
tttgcctttctctccacaggccaccatgcgggcactcgttgtaatc

SEQ ID NO: 55 - DNA, 48 bp, synthetic construct
  source 1..48: mol_type = other DNA
gtggtttgtccaaactcatcctattattcgaccttcctcttcttcttc

SEQ ID NO: 56 - DNA, 38 bp, synthetic construct
  source 1..38: mol_type = other DNA
ccgtcgtcaggatcatccggccaccatggtgagcaagg

SEQ ID NO: 57 - DNA, 44 bp, synthetic construct
  source 1..44: mol_type = other DNA
gtcgacaagccggccggtactattacttgtacagctcgtccatg

SEQ ID NO: 58 - DNA, 38 bp, synthetic construct
  source 1..38: mol_type = other DNA
aggatccgctagcctgcaggcattgtgctaagtgaaac

SEQ ID NO: 59 - DNA, 39 bp, synthetic construct
  source 1..39: mol_type = other DNA
gcttgccgatatcgaattcgggaaagcaaattgctgttg

SEQ ID NO: 60 - DNA, 40 bp, synthetic construct
  source 1..40: mol_type = other DNA
aggatccgctagcctgcagggcaactgtatatccacattc

SEQ ID NO: 61 - DNA, 39 bp, synthetic construct
  source 1..39: mol_type = other DNA
gcttgccgatatcgaattcggtgaccttttgtgtctgac

SEQ ID NO: 62 - DNA, 41 bp, synthetic construct
  source 1..41: mol_type = other DNA
aggatccgctagcctgcaggggaatacattgtcatgttatg

SEQ ID NO: 63 - DNA, 40 bp, synthetic construct
  source 1..40: mol_type = other DNA
gcttgccgatatcgaattcggtatgctgttggtgataatg

SEQ ID NO: 64 - DNA, 40 bp, synthetic construct
  source 1..40: mol_type = other DNA
aggatccgctagcctgcaggagcaaagtaatactctcctg

SEQ ID NO: 65 - DNA, 44 bp, synthetic construct
  source 1..44: mol_type = other DNA
gcttgccgatatcgaattcgttcataatatgaaactctaaggtg

SEQ ID NO: 66 - DNA, 38 bp, synthetic construct
  source 1..38: mol_type = other DNA
aggatccgctagcctgcaggagggaggttgcagtgagc

SEQ ID NO: 67 - DNA, 38 bp, synthetic construct
  source 1..38: mol_type = other DNA
gcttgccgatatcgaattcgaagtcactcccaactggg

SEQ ID NO: 68 - DNA, 41 bp, synthetic construct
  source 1..41: mol_type = other DNA
aggatccgctagcctgcaggtcaatactgcatgagttccgc

SEQ ID NO: 69 - DNA, 38 bp, synthetic construct
  source 1..38: mol_type = other DNA
gcttgccgatatcgaattcgaggcatcaggctggtgag

SEQ ID NO: 70 - DNA, 39 bp, synthetic construct
  source 1..39: mol_type = other DNA
aggatccgctagcctgcaggaggagccagaagtgcaaag

SEQ ID NO: 71 - DNA, 39 bp, synthetic construct
  source 1..39: mol_type = other DNA
gcttgccgatatcgaattcggatcacaggcaggagccac

SEQ ID NO: 72 - DNA, 43 bp, synthetic construct
  source 1..43: mol_type = other DNA
gatcgcccttcccaacagttgcagaatcacacagtccattttg

SEQ ID NO: 73 - DNA, 39 bp, synthetic construct
  source 1..39: mol_type = other DNA
caatgtcgatcgacgatgattcaccggtaacaggtgctg

SEQ ID NO: 74 - DNA, 39 bp, synthetic construct
  source 1..39: mol_type = other DNA
gtatcttatcatgtctgcggtttgtgttcccccagaaag

SEQ ID NO: 75 - DNA, 39 bp, synthetic construct
  source 1..39: mol_type = other DNA
gccaagcttgcatgcctgcactgaagctggagcaacatg

SEQ ID NO: 76 - DNA, 18 bp, synthetic construct
  source 1..18: mol_type = other DNA
cattgtgctaagtgaaac

SEQ ID NO: 77 - DNA, 20 bp, synthetic construct
  source 1..20: mol_type = other DNA
gcaactgtatatccacattc

SEQ ID NO: 78 - DNA, 21 bp, synthetic construct
  source 1..21: mol_type = other DNA
ggaatacattgtcatgttatg

SEQ ID NO: 79 - DNA, 20 bp, synthetic construct
  source 1..20: mol_type = other DNA
agcaaagtaatactctcctg

SEQ ID NO: 80 - DNA, 15 bp, synthetic construct
  source 1..15: mol_type = other DNA
ggaagaggcgctcct

SEQ ID NO: 81 - DNA, 22 bp, synthetic construct
  source 1..22: mol_type = other DNA
tccatctgcaaatgacaatatg

SEQ ID NO: 82 - DNA, 17 bp, synthetic construct
  source 1..17: mol_type = other DNA
cttgagcccaggaagtc

SEQ ID NO: 83 - DNA, 20 bp, synthetic construct
  source 1..20: mol_type = other DNA
atgggctatgaactaatgac

SEQ ID NO: 84 - DNA, 18 bp, synthetic construct
  source 1..18: mol_type = other DNA
tatgtgtgggagggctaa

SEQ ID NO: 85 - DNA, 19 bp, synthetic construct
  source 1..19: mol_type = other DNA
ggaaagcaaattgctgttg

SEQ ID NO: 86 - DNA, 19 bp, synthetic construct
  source 1..19: mol_type = other DNA
gtgaccttttgtgtctgac

SEQ ID NO: 87 - DNA, 36 bp, synthetic construct
  source 1..36: mol_type = other DNA
tcaaaatacattagaaaactatactaatcaaaacac

SEQ ID NO: 88 - DNA, 22 bp, synthetic construct
  source 1..22: mol_type = other DNA
gagacctcagaactcatctagc

SEQ ID NO: 89 - DNA, 17 bp, synthetic construct
  source 1..17: mol_type = other DNA
aggagttaggtgtgcca

SEQ ID NO: 90 - DNA, 28 bp, synthetic construct
  source 1..28: mol_type = other DNA
tgattcaatatatgttgattaattttgc

SEQ ID NO: 91 - DNA, 17 bp, synthetic construct
  source 1..17: mol_type = other DNA
gctgctgcttcttgagg

SEQ ID NO: 92 - DNA, 18 bp, synthetic construct
  source 1..18: mol_type = other DNA
cattgtgctaagtgaaac

SEQ ID NO: 93 - DNA, 19 bp, synthetic construct
  source 1..19: mol_type = other DNA
ggaaagcaaattgctgttg

SEQ ID NO: 94 - DNA, 20 bp, synthetic construct
  source 1..20: mol_type = other DNA
gcaactgtatatccacattc

SEQ ID NO: 95 - DNA, 19 bp, synthetic construct
  source 1..19: mol_type = other DNA
gtgaccttttgtgtctgac

SEQ ID NO: 96 - DNA, 21 bp, synthetic construct
  source 1..21: mol_type = other DNA
ggaatacattgtcatgttatg

SEQ ID NO: 97 - DNA, 20 bp, synthetic construct
  source 1..20: mol_type = other DNA
gtatgctgttggtgataatg

SEQ ID NO: 98 - DNA, 20 bp, synthetic construct
  source 1..20: mol_type = other DNA
agcaaagtaatactctcctg

SEQ ID NO: 99 - DNA, 24 bp, synthetic construct
  source 1..24: mol_type = other DNA
ttcataatatgaaactctaaggtg

SEQ ID NO: 100 - DNA, 18 bp, synthetic construct
  source 1..18: mol_type = other DNA
agggaggttgcagtgagc

SEQ ID NO: 101 - DNA, 18 bp, synthetic construct
  source 1..18: mol_type = other DNA
aagtcactcccaactggg

SEQ ID NO: 102 - DNA, 21 bp, synthetic construct
  source 1..21: mol_type = other DNA
tcaatactgcatgagttccgc

SEQ ID NO: 103 - DNA, 18 bp, synthetic construct
  source 1..18: mol_type = other DNA
aggcatcaggctggtgag

SEQ ID NO: 104 - DNA, 19 bp, synthetic construct
  source 1..19: mol_type = other DNA
aggagccagaagtgcaaag

SEQ ID NO: 105 - DNA, 19 bp, synthetic construct
  source 1..19: mol_type = other DNA
gatcacaggcaggagccac

SEQ ID NO: 106 - DNA, 18 bp, synthetic construct
  source 1..18: mol_type = other DNA
tgccagtggtgatgtact

SEQ ID NO: 107 - DNA, 18 bp, synthetic construct
  source 1..18: mol_type = other DNA
acagctacctccctacac

SEQ ID NO: 108 - DNA, 21 bp, synthetic construct
  source 1..21: mol_type = other DNA
ttatttctgtgtaaactttca

SEQ ID NO: 109 - DNA, 18 bp, synthetic construct
  source 1..18: mol_type = other DNA
aacctgaagtatcttgtg

SEQ ID NO: 110 - DNA, 19 bp, synthetic construct
  source 1..19: mol_type = other DNA
gtacctaacacataactgg

SEQ ID NO: 111 - DNA, 18 bp, synthetic construct
  source 1..18: mol_type = other DNA
cagtaaatactcttcccc

SEQ ID NO: 112 - DNA, 18 bp, synthetic construct
  source 1..18: mol_type = other DNA
tccactgagaatgaccct

SEQ ID NO: 113 - DNA, 18 bp, synthetic construct
  source 1..18: mol_type = other DNA
caaccttatactctggga

SEQ ID NO: 114 - DNA, 18 bp, synthetic construct
  source 1..18: mol_type = other DNA
actggaaaatgggtagac

SEQ ID NO: 115 - DNA, 18 bp, synthetic construct
  source 1..18: mol_type = other DNA
ccatggggtaggttgatt

SEQ ID NO: 116 - DNA, 18 bp, synthetic construct
  source 1..18: mol_type = other DNA
accacaagaactcaaact

SEQ ID NO: 117 - DNA, 20 bp, synthetic construct
  source 1..20: mol_type = other DNA
cccctctcattttttgtcta

SEQ ID NO: 118 - DNA, 19 bp, synthetic construct
  source 1..19: mol_type = other DNA
gaagaatatgcatagggag

SEQ ID NO: 119 - DNA, 18 bp, synthetic construct
  source 1..18: mol_type = other DNA
aggtgaataggagattgc

SEQ ID NO: 120 - DNA, 18 bp, synthetic construct
  source 1..18: mol_type = other DNA
ggtgccctttgaagattt

SEQ ID NO: 121 - DNA, 18 bp, synthetic construct
  source 1..18: mol_type = other DNA
tgggatcctcaagacatt

SEQ ID NO: 122 - DNA, 19 bp, synthetic construct
  source 1..19: mol_type = other DNA
agccatttagtcagtacag

SEQ ID NO: 123 - DNA, 20 bp, synthetic construct
  source 1..20: mol_type = other DNA
tactatgagagtgttgtgag

SEQ ID NO: 124 - DNA, 18 bp, synthetic construct
  source 1..18: mol_type = other DNA
gtgtcaaggggataatct

SEQ ID NO: 125 - DNA, 19 bp, synthetic construct
  source 1..19: mol_type = other DNA
ttgtgtatggacaggagga

SEQ ID NO: 126 - DNA, 17 bp, synthetic construct
  source 1..17: mol_type = other DNA
cagattctgacagaagg

SEQ ID NO: 127 - DNA, 22 bp, synthetic construct
  source 1..22: mol_type = other DNA
atactaaagacagagaatgtta

SEQ ID NO: 128 - DNA, 18 bp, synthetic construct
  source 1..18: mol_type = other DNA
tacccaaaggctctaacc

SEQ ID NO: 129 - DNA, 18 bp, synthetic construct
  source 1..18: mol_type = other DNA
acatgatcctcctctctt

SEQ ID NO: 130 - DNA, 19 bp, synthetic construct
  source 1..19: mol_type = other DNA
gaccaagaaacaatagacc

SEQ ID NO: 131 - DNA, 18 bp, synthetic construct
  source 1..18: mol_type = other DNA
gcttgtctattcctcttg

SEQ ID NO: 132 - DNA, 18 bp, synthetic construct
  source 1..18: mol_type = other DNA
aagagaatgggacatagc

SEQ ID NO: 133 - DNA, 18 bp, synthetic construct
  source 1..18: mol_type = other DNA
tctcacctcaatcatagc

SEQ ID NO: 134 - DNA, 19 bp, synthetic construct
  source 1..19: mol_type = other DNA
caggttttttccttcagca

SEQ ID NO: 135 - DNA, 18 bp, synthetic construct
  source 1..18: mol_type = other DNA
ttaatcatggcagcaggg

SEQ ID NO: 136 - DNA, 1500 bp, synthetic construct
  source 1..1500: mol_type = other DNA
atgcgggcactcgttgtaatcaggttgtctcgagttacggatgcgaccacgtccccggagagacaactcgaatcatgccagcagctttgtgctcagagaggatgggacgttgtgggcgtggccgaagatctcgacgtatcaggggcggtggaccccttcgatagaaagagaagaccaaatcttgccaggtggctggctttcgaagagcaaccttttgacgtaatcgtggcgtatagagtagacaggctcacgaggtctatccggcaccttcaacaattggttcactgggcagaagaccacaagaagttggtggtttcagcaactgaggcgcacttcgatactaccactccatttgcggcagtcgtgattgcccttatgggtacggttgcccaaatggaactggaggctattaaggagcggaataggagtgccgcacattttaatatccgggctggcaaatatcgcggatcactccccccgtggggttacctccctacccgcgtagacggtgagtggaggctcgtaccggaccccgttcagagagagcggatcttggaagtgtatcacagagtagtggacaaccatgaacctttgcatctcgtagcgcatgatctcaatcgcaggggggtactctctccaaaagattatttcgcgcaacttcagggtcgggagccccagggaagagaatggtctgcaacagctctcaagcggagtatgataagtgaagccatgcttggatacgccactcttaacgggaaaaccgtcagagacgacgatggcgcaccactcgtcagggcggagcccattttgacacgggaacaacttgaggcgcttcgggcagaactggttaagaccagtcgcgctaagcctgcagtgtctactccctccctcttgctcagagtcctgttttgcgcagtttgtggtgagcccgcctataaatttgctggaggcggacggaagcacccaagataccgctgcagaagcatgggatttcccaaacactgcgggaatggaacagtcgctatggccgagtgggacgccttttgcgaagagcaggtgcttgatttgctgggagacgctgaacggctggaaaaagtgtgggttgcagggtcagatagtgccgtagagttggcggaggttaacgcagaacttgtggatctgacttctttgattggctcccccgcgtatcgggccggaagcccccaacgagaagcccttgacgcccgaattgcggccctcgcagcccgacaagaggaactggaaggacttgaagcccgccctagtggatgggagtggcgggagacaggacaaaggtttggagactggtggagagaacaagatactgcggcgaagaacacatggctgcgatcaatgaacgtccggctgacgtttgacgttcggggcggcttgacaaggactattgatttcggggatctgcaagaatatgaacagcatcttagactgggctctgtagttgaaaggcttcacaccggtatgtcc

SEQ ID NO: 137 - DNA, 1560 bp, synthetic construct
  source 1..1560: mol_type = other DNA
atgcgggcactcgttgtaatcaggttgtctcgagttacggatgcgaccacgtccccggagagacaactcgaatcatgccagcagctttgtgctcagagaggatgggacgttgtgggcgtggccgaagatctcgacgtatcaggggcggtggaccccttcgatagaaagagaagaccaaatcttgccaggtggctggctttcgaagagcaaccttttgacgtaatcgtggcgtatagagtagacaggctcacgaggtctatccggcaccttcaacaattggttcactgggcagaagaccacaagaagttggtggtttcagcaactgaggcgcacttcgatactaccactccatttgcggcagtcgtgattgcccttatgggtacggttgcccaaatggaactggaggctattaaggagcggaataggagtgccgcacattttaatatccgggctggcaaatatcgcggatcactccccccgtggggttacctccctacccgcgtagacggtgagtggaggctcgtaccggaccccgttcagagagagcggatcttggaagtgtatcacagagtagtggacaaccatgaacctttgcatctcgtagcgcatgatctcaatcgcaggggggtactctctccaaaagattatttcgcgcaacttcagggtcgggagccccagggaagagaatggtctgcaacagctctcaagcggagtatgataagtgaagccatgcttggatacgccactcttaacgggaaaaccgtcagagacgacgatggcgcaccactcgtcagggcggagcccattttgacacgggaacaacttgaggcgcttcgggcagaactggttaagaccagtcgcgctaagcctgcagtgtctactccctccctcttgctcagagtcctgttttgcgcagtttgtggtgagcccgcctataaatttgctggaggcggacggaagcacccaagataccgctgcagaagcatgggatttcccaaacactgcgggaatggaacagtcgctatggccgagtgggacgccttttgcgaagagcaggtgcttgatttgctgggagacgctgaacggctggaaaaagtgtgggttgcagggtcagatagtgccgtagagttggcggaggttaacgcagaacttgtggatctgacttctttgattggctcccccgcgtatcgggccggaagcccccaacgagaagcccttgacgcccgaattgcggccctcgcagcccgacaagaggaactggaaggacttgaagcccgccctagtggatgggagtggcgggagacaggacaaaggtttggagactggtggagagaacaagatactgcggcgaagaacacatggctgcgatcaatgaacgtccggctgacgtttgacgttcggggcggcttgacaaggactattgatttcggggatctgcaagaatatgaacagcatcttagactgggctctgtagttgaaaggcttcacaccggtatgtccaaaaggaccgcagatggctccgagtttgagtcaccgaagaagaagaggaaggtcgaataa

SEQ ID NO: 138 - DNA, 554 bp, Homo sapiens
  source 1..554: mol_type = genomic DNA
gtggatgaatgaataaagatgatgtggtatgtacatacaatggaatattagccttggaaaagaaggaaatactttcacatgctacaacatggatgaaccttgaggacattgtgctaagtgaaacaagccagttgcaaaaagagaatctgcttgcatggagtatgtaaaaaagtcatattcttggaaagtagaatggacattgccaggggaggaagtgggaagttcaatggatgcagagttttagttttgcaagatgaaaagttcctagggatctattgcacaacaatgtgcatgtagttgacactactgaactgtgtattgaaaaagggttaagattgcaaaaatcaatcttaggcaatttgatttttcaagacacagacatctacaaatgttattattgaagaaaccaatgcatcattacggattatgcctttggtagtgtctccaggtgcaaggaataacagaaataaaagtcacatgatgtgtgaaccccagagctttggggagactcctataccaggtctgaagtgacctaataggaagtagctgttatt

SEQ ID NO: 139 - DNA, 1665 bp, Homo sapiens
  source 1..1665: mol_type = genomic DNA
ccacaaagttttggggtaaattgttgcacagcaataaataactcgaatgaaaatattctgattaagtctttctgatgctaatttgactttgcttcttccaaaaattagtccaaatgagtaaaatgatcatttcctcttggcatgtctatagattttctacttggcatcttctccatctgcaaatgacaatatgcctttggttttctggtattccatggaagctacatgtgtcaagaagtcaatactgcatgagttccgcaaataaaccatcccactcaccatcactgttcatgcatttcctacaacagcctatatctagttcatccatgtccgcttctatcttttctctcccaggtttctgcagcaatcatactctcatttctttctaggggctctaacttctgcttctctgcttgtaggcctatgtccttctcttctgttaatgacattgggcttttccccctctttatgcacctgcagtcaaggtgggaaacaaaaagattcgaaactattttgactcagacctaggaacttgtaatgtcaccataagatgagattcctgtctccagccaaatctcaccttgaattgtaataatccctatgtgtcaacggaggagccaggtagagataattgaattataggggtggtttcccccatactgttctcatggtagtgaataagtctcacaagatctgatggttctataaatggaagatcccccgcacaagctctcttgcctgctaccatgtaagacatgcctttgcttctcctttaccttctgccatgattgtaaggcctccccagccaggtggaattgtgagtccattaagcctcttttatatatatataaattacccagtctcagatatgtctttattaacagtgtgagaacagactacaacctgaccctgacaatccttgtgcttccctcatcttgcctcttctatttccccactctgcctctgattagagagaggaatgcttctggggtaactgctaattgaaagtaggttagtctcttccacatagcgagagctaatcacccatgatccacaattccaggagcatgaacatgtatctccctcaccagcctgatgcctgaaattcaaatgcaagagattcattccttgaaattagatattggctctaaagattcttttaaagtgggaggtggccagaatgagatgattgctatagtcaggtttaacaatcaactgaaataccccagaatctgaaaaggttacctctaagaaggaaaactagtaagtgaattctatcaacatgcaaaattaatcaacatatattgaatcatactagctgctttggtaaacatcttatcctagagttttaatagtgttctaaaaagttgtctataaaggtgcacccgaggctgggcacaatggctcctgactgtaatcccagcaatttgggaggccagggaaggtggataacttgaggccaggtgtttgagatcagcctggggaacacagagaaaccccatctctactaaaaaacagaaaaattagcagggtgctgtggcgcgcacctgtagtcccagctactcgggaggctgaggcacaagaatcgcttgagcctgggagatggaggttgcagtgaaccgagatcacgccactgccctccagcctgggcgacagagtgagacg

SEQ ID NO: 140 - DNA, 1379 bp, Homo sapiens
  source 1..1379: mol_type = genomic DNA
accaaggaccagtggtgggaatgagcttggagacacggacacacagaatcagagacagaccgaaacaaagagacagagacaggcgctcagggacaccctaacagagacagacagacaaagagacaagggtctctcattggccggcattatgggttacatcgtctctcccccaaaaaacctatatgctgaagtcctcacccctcagaacctccgaatgtactggaggtggtacagatgtaactggagtagggtgggctctaatctaatacgactgatgaccttataaaaggggacaatgtggttcatgcctgtaatcccagcactttgggaggctgaggcgggggggatcacttgaggtcaggagttcgagaccagcctggccaacatggcaaaaccctgtctctactaaaaatagaaaaattagctgggtgtggtcgtgggcgcctgtaatccaagctactccagaggctgaggcaggagaatcccttgaacctgggaggcggaggttgcagtgagctgaaatcgtgccattgctctccagcctgggcaacagagtgagaagattccttctcaaaacaaacaaaaaaacgaaggggcagggggacaatgtgggcacaggtatgcgtgcagggagaatgctgtgtgaatatgaagttgccatctacaaaccaaggagaaaggcctgggacagacccctcccttacatccctcaagaagcagcagccggccaggcatggtggctcctgcctgtgatcccagtgcttttcagaggctgagatgggaggatgcttgaggccacgagctcaagaccagcctgggcaacagaatgagacctcgtctctattaaaagtaaaaaaaaaaaaaaaaaaaaaaaatacccaagtgtggtggcacaggcttgtggtcccagctactcaggaaggtgaggtggaggatcacttgagcccacgaggtcgaggctgcagtgagctgtgattgcatcattgcactccagcctgggtgacagagcaagaccttgtctcagaaaactgaaagaaagggaaggaaggaaggaaggaagggagggacgaagggaaggagggagggagggaaggaggaaggaaggaggaagagaggcagggaggaagggaaggaaggagggcaggaagaaggaagagagggttgggaggagggaagaaggaagaggggggagggaggcaggggggaaggagggaaggaaggaaggaaagaaggaaggaaggaaagaagggagggaggaaaggaaggaggaagagaagggagggaggaaagaaggaaggaaggaaggaaaaaagaaccagccctgctggcaacttggtttcagacttctagcctccaggactgtgacatatttctgtttt